SEQUENCE LISTING (4 sequences)

SEQ ID NO: 1 - protein, 1218 aa, Drosophila melanogaster
  Protein sequence of transcript hppy-RA for the gene CG7097 (happyhour)
MAAAHSHHNANMLSSDISRRNPQDEYELIQKIGSGTYGDVYKAKRIQSNELAAIKVIKLEPSDDIQIIQQEIIMMRDCRHPNIIAYYGSYLRRDKLWICMEFCGGGSLQDIYQVTGPLTEVQIAYMCRETLKGLEYLHSMGKMHRDIKGANILLTEYGDVKLADFGVSAQITATINKRKSFIGTPYWMAPEVAAVERKGGYNQLCDIWACGITAIELAELQPPMFDLHPMRALFLMSKSGFKPPTLNNKDKWSPTFHNFIKTALTKNPKKRPTAERLLQHPFVQCEMSLRVAKELLQKYQSPNPQFYYYLDGDEESVAGVPQRIASKMTSRTNGVPAQNHTLKTGMTTNSTWNERSSSPETLPSDMSLLQYIDEELKLRATLPLNNDTKDPLGAECSCSSHNGGAAGGGGGGGVGVGAGGAAANGSSSSSGGATVGTTHHQHQQHHQHHHHPNHLHQHQSHQLPQQQQQQSHQAAQQEHHHHHPMTSSISSGLVTANANNISSSASLASMPGLSAYLGMRSHVVGHMGMGFGMGLGMSNLRTTSIDADDDELVAADVAMNNAAAAVPGNGSGAGNGSGSGCSASAAHYLRYSSNYRSAAAAQASQAAHPHVNSNSNSNNGHGHAPITSSISSSASSASFYNRLLLLDNSSGDAVVGGNGGGSSNISSSGGASSGTNGLLDKHELDALPQAATKSAGDGFSHSNSPTANSGAGATGSRDNDGPSSSNSSHLYQNLLRSSSGETPAGSSSAGNNCDYRHENNQNGLEDSPRRHSSMDQLIGLLENMGKSPRTRSLSDGGTQDDDEAEKEAQPDLLNNTPPVPPKRSHKRRHTPPRPISNGLPPTPKVHMGACFSKIFNGCPLRVHCTASWIHPETRDQHLLIGAEEGIYNLNMNELHDAAIDQLFPRRTTWLYVIKDVLMSLSGKSCQLYRHDLVALHSKQTVRFSLHMNKIPERLVPRKFALTTKVPDTKCCTQCCVTRNPYNGYKYLCGATPSGIFLMQWYDPLNKFMLLKQCEWPAISIQGGGHGCVQNGHTPVFEMIITPELEYPIVCTGVRKAMNGCLKLELINMNSASWFHSEDLEYDAMATMVPRRDLLKVVRVHQVEKDAILVCYGNLIQVVTLQGNPKQHKKMVSQLNFDFNVDSIVCLPDSVLAFHKHGMQGKSLRNGEVTQEIKDMSRTYRLLGSDKVVALESQLLRTGSLGSEEGHDLYILAGHEASY

SEQ ID NO: 2 - DNA, 5071 bp, Drosophila melanogaster
  Nucleotide sequence of transcript hppy-RA for the gene CG7097 (happyhour)
catttgtgggctgcgcgttgacgaagtgcatgtgaaatttttcgtgttcgtaggacaattgttaattatccactggccgaatttgccccgcaatcacgtgtcggtgtaatatgtgaacgcgtccaaaatgcatctcgatccacaaaagtatctaaatttcgcctgctacgtgtgctaatcagtcaaacccaaaaaacgaataaagcagcgtcaagagtaagagtgtcaatcaaatattaagagtatacccacccagtaaagagaggtccagccaaaaggactcgatcgcctcgaaccgaaagcggaagcggaagcggaaacggcaacggcatacggatacggaagctgccaagatggccgccgcccacagtcaccacaatgccaatatgctcagctcggacatctctcgccgaaatccgcaggacgagtacgaactcatccaaaagattggctcgggaacctacggcgacgtctataaggcgaaacgaatacagagcaatgagctggctgccatcaaggtcatcaagctggagccttccgacgatatacagattatccagcaggagattatcatgatgagggactgccgccatccgaacatcatcgcctactacggttcgtacctgcgcagggataaactctggatctgcatggagttttgtggtggtggcagtctgcaggacatttaccaagtgaccggtcccctcaccgaagtccagatcgcgtacatgtgccgagagactctcaagggactcgagtacctgcactccatgggaaaaatgcaccgggacatcaagggcgcaaatatcctgttgacggagtacggcgatgtcaagctggcggatttcggcgtttcagctcagatcacggccacaattaacaaacgcaagagcttcataggtacgccctactggatggctcctgaggtggcagccgtggaacgcaagggtggctataatcaactatgtgatatttgggcctgcggcataaccgcaatcgaactggctgaactgcaaccgccgatgttcgatttgcacccgatgcgcgccctattcctgatgtcgaagagcggctttaagccgcccactctgaacaacaaggacaagtggagccccacgttccacaacttcatcaagacggcgctgacgaagaatccgaagaagcgacccaccgccgagcgcctgctgcagcatcccttcgtccagtgcgagatgtccttgcgggtggccaaggagctgctgcagaagtaccagagtcccaacccgcagttctactactatctcgatggcgatgaggagtctgtggcaggagtgccacaacgcattgccagcaaaatgacgtcacgcaccaatggcgtgccagcgcaaaatcacacactaaaaacaggcatgacgacgaactccacgtggaatgagcgatcttctagtcccgaaacgttacccagtgacatgagcctcttacaatatattgatgaggagctgaagctaagagcgaccttgccactgaacaacgacaccaaagatccactcggcgccgagtgcagctgctcctcccacaatggaggagccgccggaggaggaggaggaggaggagttggagtaggagcaggcggagcagccgcgaacggcagcagcagcagcagcggaggcgcaacagtcggcaccactcatcatcagcaccaacagcaccaccagcatcaccaccatccgaatcatctgcatcagcatcagtcccatcaattgccgcaacagcagcagcagcagtcacatcaggcggcacagcaggaacaccatcaccaccatccaatgacctcgtccatatccagcggcttggtgaccgccaatgccaacaacatcagctcgtccgcctcgctggcctccatgccaggtctcagtgcctacctgggcatgcgctcccatgtggttggccacatgggtatgggatttggcatgggtctcggcatgtccaacctgcgcaccaccagcatcgatgccgacgacgatgaactggtggctgccgatgtggccatgaataatgctgctgccgccgttcctgggaacggatccggtgctggcaacggttccggatccggatgctccgcctctgcggcgcactatctgcgttacagcagcaactaccgatccgccgccgcggctcaggcatcgcaggcggcacatccgcacgttaactccaattcgaattcgaacaacggccatggccatgcgccgatcacatccagcatctcgtcctctgcgtcatcagcttcgttttataatcgcctcctactcctcgataattccagcggcgatgcagtcgtaggcggtaacggaggcggcagcagcaacataagcagcagcggaggagcatcaagtggcaccaatggcctcctggataagcacgagctagatgctcttccccaagcggcaacaaagtcggccggcgatggcttctcgcattccaatagcccaacggcgaattccggtgcgggagcgaccggatccagggataacgatggaccctcgtcgagcaactcttcgcatctctaccagaacctgctgagaagcagctctggtgaaacgccagcgggatcgagctcggcgggcaataactgtgattaccggcatgagaacaaccagaatggtctggaggactcgcctcgacgccatagctccatggatcagctgattgggttgctcgagaacatgggcaagtctccgaggacacgcagcctgagcgatggtggcacccaggacgatgacgaagcggagaaggaggcgcaaccggatctgctgaacaacaccccaccggtaccaccgaagcgctcccacaaacgccgtcacacgccacctcgacccatctccaacggactgccgcccacacccaaggtgcacatgggcgcctgcttctccaagatcttcaacggttgcccactgcgcgtccactgcaccgcctcgtggattcatccggagacgcgggaccagcatctgctgatcggcgccgaggagggcatctacaacctcaacatgaacgaactgcatgacgcggccatcgatcagttgttcccgcgccgcaccacctggttgtatgtcatcaaggatgtgctcatgagtttgtcaggaaaatcctgccagctctacaggcacgacctggtggccctgcactccaagcagacggtacgcttctcgctgcacatgaacaagatcccggagcgactggtgccgcgcaagttcgctctcaccaccaaggtgccggacacaaagtgctgcacacagtgctgtgtcacacggaatccgtataacggatacaaatatctctgtggggcgacacccagcggcattttcctgatgcagtggtacgatccactgaacaagtttatgctgctcaagcagtgcgagtggccagccatcagcattcagggtggtggtcatggctgcgttcagaacggacatacgcccgtattcgagatgattatcacgccggagttggagtacccgattgtgtgtacgggtgtgcgaaaggccatgaacggctgcctcaagctggagctgatcaacatgaacagtgccagctggttccattcggaggacctggaatatgacgccatggccacgatggtgccgcgacgcgacctgctgaaggtggtgcgtgtgcaccaggtggagaaggacgccattctggtctgctacggcaatctgattcaggtggtcaccctgcagggcaatcccaagcagcacaagaagatggtatcgcagctcaactttgatttcaacgtggacagcattgtttgtctaccggacagcgttttggcattccataagcacggcatgcagggaaagtcgttgcgcaatggcgaggtgacgcaggagatcaaggacatgagtcgcacctacaggctgctgggcagcgataaggttgtggccttggagagccagctgctgaggaccggctccctgggcagcgaggagggacacgatctgtacattctggccgggcacgaggctagctactaagctaatgggcgcccgcccaatccactgttatattttaaatataatccggttcaccacacacattttaaagggctgtcgccgccgtcgtcgtcaactattaactgtaatttaattactcgtaggctaagcagtgagttcactgatcatagatgactagagagagccagttgcatgccgagcgttcgatagcttagattagcgcaatttaacagcacctagaagcggcggtcttcgaagaagggttccacactaccatgcacactgacgggtatactcgacccgaatcgctatctttagatctgccgatcactcgatatatattgaacgtctgcgtctacccgattccgttgtagctatatttttgtattacaaagaaatgctttaatttatacaactaaccttaatgaactacagtaatcgctacgtctagttatgttagagatttgtgtaaacgcttcacaaagccaatagatacgttttcaaatagtcagccagcaggagaatccaagtatacatacacttaaacacgcaattatccacacgattcgagaccgactaaggcaattagaactaaacaattatatttagtctatagcaattgtaaatcgcaatgtttttagctggttaaactgctttcgattggctcaaaaccgcaacggaaaatcaagaacgagaaattgaagaatgcgatataggatgatacggtttgttgggaaacaaaactagttagtaagacttttctaacctagtaacacgaattcaaaacaaaataaatgcaaattaatggtctgtatctgtatgttaaaaaagtaaactgattgtcatagttacggttctaggctataagataagcgatgaaaacagacaactttccgaacagcacatttgtcgattgagttgtataaatattctactagatgctaagttatttagttcatacgacaacaacaaagcaaaaaccgaaaattacaaactaatacggagaaaaacaaacagataaactcacaaaattgttgaactctagcttagtgcaaaaataaaaaaaaaatacgtgaaagaaa

SEQ ID NO: 3 - protein, 947 aa, Drosophila melanogaster
  Protein sequence of transcript hppy-RB for the gene CG7097 (happyhour)
MAAAHSHHNANMLSSDISRRNPQDEYELIQKIGSGTYGDVYKAKRIQSNELAAIKVIKLEPSDDIQIIQQEIIMMRDCRHPNIIAYYGSYLRRDKLWICMEFCGGGSLQDIYQVTGPLTEVQIAYMCRETLKGLEYLHSMGKMHRDIKGANILLTEYGDVKLADFGVSAQITATINKRKSFIGTPYWMAPEVAAVERKGGYNQLCDIWACGITAIELAELQPPMFDLHPMRALFLMSKSGFKPPTLNNKDKWSPTFHNFIKTALTKNPKKRPTAERLLQHPFVQCEMSLRVAKELLQKYQSPNPQFYYYLDGDEESVAGVPQRIASKMTSRTNGVPAQNHTLKTGMTTNSTWNERSSSPETLPSDMSLLQYIDEELKLSGDAVVGGNGGGSSNISSSGGASSGTNGLLDKHELDALPQAATKSAGDGFSHSNSPTANSGAGATGSRDNDGPSSSNSSHLYQNLLRSSSGETPAGSSSAGNNCDYRHENNQNGLEDSPRRHSSMDQLIGLLENMGKSPRTRSLSDGGTQDDDEAEKEAQPDLLNNTPPVPPKRSHKRRHTPPRPISNGLPPTPKVHMGACFSKIFNGCPLRVHCTASWIHPETRDQHLLIGAEEGIYNLNMNELHDAAIDQLFPRRTTWLYVIKDVLMSLSGKSCQLYRHDLVALHSKQTVRFSLHMNKIPERLVPRKFALTTKVPDTKCCTQCCVTRNPYNGYKYLCGATPSGIFLMQWYDPLNKFMLLKQCEWPAISIQGGGHGCVQNGHTPVFEMIITPELEYPIVCTGVRKAMNGCLKLELINMNSASWFHSEDLEYDAMATMVPRRDLLKVVRVHQVEKDAILVCYGNLIQVVTLQGNPKQHKKMVSQLNFDFNVDSIVCLPDSVLAFHKHGMQGKSLRNGEVTQEIKDMSRTYRLLGSDKVVALESQLLRTGSLGSEEGHDLYILAGHEASY

SEQ ID NO: 4 - DNA, 4258 bp, Drosophila melanogaster
  Nucleotide sequence of transcript hppy-RB for the gene CG7097 (happyhour)
catttgtgggctgcgcgttgacgaagtgcatgtgaaatttttcgtgttcgtaggacaattgttaattatccactggccgaatttgccccgcaatcacgtgtcggtgtaatatgtgaacgcgtccaaaatgcatctcgatccacaaaagtatctaaatttcgcctgctacgtgtgctaatcagtcaaacccaaaaaacgaataaagcagcgtcaagagtaagagtgtcaatcaaatattaagagtatacccacccagtaaagagaggtccagccaaaaggactcgatcgcctcgaaccgaaagcggaagcggaagcggaaacggcaacggcatacggatacggaagctgccaagatggccgccgcccacagtcaccacaatgccaatatgctcagctcggacatctctcgccgaaatccgcaggacgagtacgaactcatccaaaagattggctcgggaacctacggcgacgtctataaggcgaaacgaatacagagcaatgagctggctgccatcaaggtcatcaagctggagccttccgacgatatacagattatccagcaggagattatcatgatgagggactgccgccatccgaacatcatcgcctactacggttcgtacctgcgcagggataaactctggatctgcatggagttttgtggtggtggcagtctgcaggacatttaccaagtgaccggtcccctcaccgaagtccagatcgcgtacatgtgccgagagactctcaagggactcgagtacctgcactccatgggaaaaatgcaccgggacatcaagggcgcaaatatcctgttgacggagtacggcgatgtcaagctggcggatttcggcgtttcagctcagatcacggccacaattaacaaacgcaagagcttcataggtacgccctactggatggctcctgaggtggcagccgtggaacgcaagggtggctataatcaactatgtgatatttgggcctgcggcataaccgcaatcgaactggctgaactgcaaccgccgatgttcgatttgcacccgatgcgcgccctattcctgatgtcgaagagcggctttaagccgcccactctgaacaacaaggacaagtggagccccacgttccacaacttcatcaagacggcgctgacgaagaatccgaagaagcgacccaccgccgagcgcctgctgcagcatcccttcgtccagtgcgagatgtccttgcgggtggccaaggagctgctgcagaagtaccagagtcccaacccgcagttctactactatctcgatggcgatgaggagtctgtggcaggagtgccacaacgcattgccagcaaaatgacgtcacgcaccaatggcgtgccagcgcaaaatcacacactaaaaacaggcatgacgacgaactccacgtggaatgagcgatcttctagtcccgaaacgttacccagtgacatgagcctcttacaatatattgatgaggagctgaagctaagcggcgatgcagtcgtaggcggtaacggaggcggcagcagcaacataagcagcagcggaggagcatcaagtggcaccaatggcctcctggataagcacgagctagatgctcttccccaagcggcaacaaagtcggccggcgatggcttctcgcattccaatagcccaacggcgaattccggtgcgggagcgaccggatccagggataacgatggaccctcgtcgagcaactcttcgcatctctaccagaacctgctgagaagcagctctggtgaaacgccagcgggatcgagctcggcgggcaataactgtgattaccggcatgagaacaaccagaatggtctggaggactcgcctcgacgccatagctccatggatcagctgattgggttgctcgagaacatgggcaagtctccgaggacacgcagcctgagcgatggtggcacccaggacgatgacgaagcggagaaggaggcgcaaccggatctgctgaacaacaccccaccggtaccaccgaagcgctcccacaaacgccgtcacacgccacctcgacccatctccaacggactgccgcccacacccaaggtgcacatgggcgcctgcttctccaagatcttcaacggttgcccactgcgcgtccactgcaccgcctcgtggattcatccggagacgcgggaccagcatctgctgatcggcgccgaggagggcatctacaacctcaacatgaacgaactgcatgacgcggccatcgatcagttgttcccgcgccgcaccacctggttgtatgtcatcaaggatgtgctcatgagtttgtcaggaaaatcctgccagctctacaggcacgacctggtggccctgcactccaagcagacggtacgcttctcgctgcacatgaacaagatcccggagcgactggtgccgcgcaagttcgctctcaccaccaaggtgccggacacaaagtgctgcacacagtgctgtgtcacacggaatccgtataacggatacaaatatctctgtggggcgacacccagcggcattttcctgatgcagtggtacgatccactgaacaagtttatgctgctcaagcagtgcgagtggccagccatcagcattcagggtggtggtcatggctgcgttcagaacggacatacgcccgtattcgagatgattatcacgccggagttggagtacccgattgtgtgtacgggtgtgcgaaaggccatgaacggctgcctcaagctggagctgatcaacatgaacagtgccagctggttccattcggaggacctggaatatgacgccatggccacgatggtgccgcgacgcgacctgctgaaggtggtgcgtgtgcaccaggtggagaaggacgccattctggtctgctacggcaatctgattcaggtggtcaccctgcagggcaatcccaagcagcacaagaagatggtatcgcagctcaactttgatttcaacgtggacagcattgtttgtctaccggacagcgttttggcattccataagcacggcatgcagggaaagtcgttgcgcaatggcgaggtgacgcaggagatcaaggacatgagtcgcacctacaggctgctgggcagcgataaggttgtggccttggagagccagctgctgaggaccggctccctgggcagcgaggagggacacgatctgtacattctggccgggcacgaggctagctactaagctaatgggcgcccgcccaatccactgttatattttaaatataatccggttcaccacacacattttaaagggctgtcgccgccgtcgtcgtcaactattaactgtaatttaattactcgtaggctaagcagtgagttcactgatcatagatgactagagagagccagttgcatgccgagcgttcgatagcttagattagcgcaatttaacagcacctagaagcggcggtcttcgaagaagggttccacactaccatgcacactgacgggtatactcgacccgaatcgctatctttagatctgccgatcactcgatatatattgaacgtctgcgtctacccgattccgttgtagctatatttttgtattacaaagaaatgctttaatttatacaactaaccttaatgaactacagtaatcgctacgtctagttatgttagagatttgtgtaaacgcttcacaaagccaatagatacgttttcaaatagtcagccagcaggagaatccaagtatacatacacttaaacacgcaattatccacacgattcgagaccgactaaggcaattagaactaaacaattatatttagtctatagcaattgtaaatcgcaatgtttttagctggttaaactgctttcgattggctcaaaaccgcaacggaaaatcaagaacgagaaattgaagaatgcgatataggatgatacggtttgttgggaaacaaaactagttagtaagacttttctaacctagtaacacgaattcaaaacaaaataaatgcaaattaatggtctgtatctgtatgttaaaaaagtaaactgattgtcatagttacggttctaggctataagataagcgatgaaaacagacaactttccgaacagcacatttgtcgattgagttgtataaatattctactagatgctaagttatttagttcatacgacaacaacaaagcaaaaaccgaaaattacaaactaatacggagaaaaacaaacagataaactcacaaaattgttgaactctagcttagtgcaaaaataaaaaaaaaatacgtgaaagaaa

(19) United States
(12) Patent Application Publication
(10) Pub. No.: US 2009/0087876 A1
(43) Pub. Date: Apr. 2, 2009

(54) METHODS AND COMPOSITIONS USEFUL FOR MODULATING DRUG-INDUCED IMPAIRMENT

(76) Inventors: Ulrike Heberlein, Hillsborough, CA (US); Karen Berger, El Cerrito, CA (US); Ammon Ben Corl, San Francisco, CA (US)

Correspondence Address: JONES DAY, 222 EAST 41ST ST, NEW YORK, NY, 10017, US

(21) Appl. No.: 12/172,918

(22) Filed: Jul. 14, 2008

Related U.S. Application Data
(60) Provisional application No. 60/959,474, filed on Jul. 13, 2007.

(51) Int. Cl. C12Q 1/02; C07H 21/04; C07K 14/435; C12N 15/63; C12N 5/10; C07K 16/18

(52) U.S. Cl. 435/29; 536/23.5; 530/350; 435/320.1; 435/325; 530/387.1; 530/389.1; 530/388.2

(57) ABSTRACT

The present invention provides isolated nucleic acids, polypeptides, oligonucleotides, vectors, host cells, antibodies, compositions, and kits relating to happyhour. Also provided are methods of screening for agents capable of modulating happyhour activity.

### 1. RELATED APPLICATION

This application is related to U.S. Provisional Patent Application No. 60/959,474, filed Jul. 13, 2007 and entitled “Methods and Compositions Useful for Modulating Drug-induced Impairment,” the entire contents of which are hereby incorporated by reference herein.

### 2. FIELD OF THE INVENTION

The present invention relates to happyhour, a modulator of the EGFR/ERK signaling pathway. In certain aspects, the present invention provides isolated happyhour-encoding nucleic acids, isolated happyhour polypeptides, oligonucleotides that hybridize to happyhour nucleic acids, and expression vectors containing happyhour polypeptide-encoding sequences. The present invention further provides isolated host cells, antibodies, compositions, and kits relating to happyhour. In other aspects, the present invention further provides methods of screening for agents that modulating happyhour activity. The agents are useful, for example for modulating drug-induced impairment when administered to a subject in need thereof.

### 3. BACKGROUND OF THE INVENTION

Substance/drug abuse refers to overindulgence in and dependence on a psychoactive leading to effects that are detrimental to the individuals physical health or mental health or the welfare of others. Examples of well-known psychoactives are cocaine, ethanol, LSD, cannabis etc. It is thought that there may be substantial overlap and downstream convergence, for example, in the positive reinforcing effects of multiple drugs in the mesolimbic dopamine system. Thus, pharmacotherapies designed against targets of ethanol's actions may generalize to other drugs of abuse.

Alcohol (ethanol) is one of the most popularly consumed and abused drugs in the world. The pleasurable and disinhibiting effects of alcohol consumption have been enjoyed by humankind for thousands of years. For some, however, alcohol consumption leads to alcohol addiction, a devastating illness with enormous medical and societal costs. In the United States, for example, approximately 7% of adults are alcoholics, and alcohol-related problems cost the country over $200 billion per year, and are responsible for 100,000 deaths (Diamond, and Gordon, 1997, Physiological Reviews 77:1-20; Volpicelli, 2001, J Clin Psychiatry 62 Suppl 20:4-10). A better understanding of the genetic and environmental factors that contribute to the development of alcoholism would provide considerable benefits to those who suffer from alcohol addiction and to society in general.

Although the cognitive and behavioral changes associated with alcohol consumption are quite familiar, knowledge concerning the mechanisms through which ethanol acts on the central nervous system to produce these behavioral changes is still far from complete. Rather than acting on a single molecular target, in vitro studies have shown that ethanol exerts effects on multiple different voltage-gated and ligand-gated ion channels. For example, ethanol has been found to potentiate the function of [symbol] -aminobutyric acid-A (GABAA) receptors and inhibit N-methyl-D-aspartate (NMDA) receptors at pharmacologically relevant ethanol concentrations (reviewed in Diamond and Gordon, 1997; Lovinger, 1997, Arch Pharmacol 356:267-282). More recently, studies of genetically engineered mice have provided further insight into some molecules that regulate the behavioral response to ethanol in vivo, demonstrating roles for serotonin, dopamine, and cannabinoid systems, as well as several signal transduction pathways (Crabbe, et al., 2006, Addict Biol 11:195-269).

Family, adoption, and twin studies strongly show a genetic component to alcoholism, although thus far attempts in humans to identify specific genes underlying alcoholism have been largely unsuccessful (Reich et al., 1999, Am Hum Genet 65:599-605; Schuckit et al., 2004, Alcohol Clin Exp Res 28:1449-1458). Interestingly, human studies also indicate that the level of response to intoxicating doses of ethanol act as a predictor of future alcoholism (Schuckit et al., 2004). For example, a low level of response to ethanol at age 20 was found to be associated with a four-fold increased likelihood of development of alcoholism within the following ten years (Schuckit, 1994, Am J Psychiatry 151:184-189). This strongly suggests that the identification of genes and pathways mediating acute responses to ethanol promises to offer helpful insight into the genetic factors contributing to alcohol addiction.

The fruit fly, Drosophila melanogaster, with its accessibility to genetic and molecular analysis, has proven itself as an attractive model system in which to study the genes and pathways that modify acute and chronic behavioral responses to ethanol exposure. (reviewed in Guarnieri and Heberlein, 2003, Int Rev Neurobiol 54:199-228; Wolf and Heberlein, 2003, J Neurobiol 54:161-178). In response to acute ethanol exposure, flies exhibit behaviors similar to those observed in mammals: low doses of ethanol result in hyperactivity, whereas higher doses result in decreased activity and eventual loss of postural control and sedation (Singh and Heberlein, 2000, Alcohol Clin Exp Res 24:1127-1136; Wolf et al., 2002, J Ncurosci 22:11035-11044). Unbiased genetic approaches and candidate gene analyses have provided insight into the various molecules and biochemical pathways (Corl et al., 2005, Nat Neurosci 8:18-19; Moore et al., 1998, Cell 93:997-1007; Park et al., 2000, J. Biol. Chem. 275:20588-20596; Wen et al., 2005, Proc Natl Acad Sci USA 102:2141-2146; Rothentluh et al., 2006, Cell 127:199-211) as well as the neuroanatomical loci (Rodan et al., 2002, J Neurosci 22:9490-9501) that regulate the ethanol response in Drosophila. Several of the molecules implicated in ethanol responses in Drosophila, such as protein kinase A (PKA), calcium-dependent adenylate cyclase (Moore et al., 1998) and the fly orthologue of neuropeptide Y, NPF (Wen et al., 2005), have been shown to have similar roles in mediating ethanol behaviors in mammals (Thiele et al., 2000, J Neurosci 20:RC75; Thiele et al., 2002, J Neurosci 22:RC208; Maas et al., 2005, J Neurosci 25:4118-4126), validating the usefulness of Drosophila as a valuable tool for identifying candidate genes and pathways underlying the behavioral response to ethanol.

Mitogen-activated protein (MAP) kinase signaling cascades have been shown to play essential roles in regulating a variety of cellular processes, including embryogenesis, cell differentiation and proliferation, cell death, and acute responses to hormones and environmental stresses (reviewed in Chen et al., 2001, Chem Rev 101:2449-2476; Pearson et al., 2001, Endocr Rev 22:153-183). Two of the major MAP kinase cascades, the c-Jun N-terminal kinase (JNK) pathway and the p38 pathway, play important roles in transducing cellular stress responses triggered by stimuli such as osmotic shock, heat shock, and inflammatory cytokines (Pearson et al., 2001). The other major and most well characterized MAP kinase pathway is the extracellular signal-regulated protein kinase (ERK) cascade, activated by growth factors, serum, and cytokines, which has been shown to play major roles not only developmentally in cell proliferation and differentiation, but also postmitotically in regulating synaptic transmission and long-term memory (Pearson et al., 2001; Sweatt, 2004, Curr Opin Neurobiol 14:311-317; Mazzucchelli and Brambilla, 2000, Cell Mol Life Sci. 57:604-11) as well as circadian rhythms (Kramer et al., 2001, Science 294:2511-2515). In Drosophila, the ERK pathway, and more specifically its activation through the epidermal growth factor (EGF) receptor (EGFR), has been implicated in various phases of development, including the specification of cell fate in the central nervous system, germ band retraction in the embryo, and the development of the retina (reviewed in Kumar et al., 1998, Development 125:3875-3885; Perrimon and Perkins, 1997, Cell 89:13-16). In recent years, studies in vitro and in vivo have revealed an intriguing link between ethanol and the mammalian EGFR/ERK pathway, demonstrating that EGFR autophosphorylation and ERK phosphorylation are both inhibited by pharmacologically relevant concentrations of ethanol (Chandler and Sutton, 2005, Alcohol Clin Exp Res 29:672-682; Ma et al., 2005, Biochem Pharmacol 69:1785-1794). While these studies have shown ethanol to act as an inhibitor of EGFR/ERK signaling in both neuronal cell cultures and mammalian brains, the roles of the EGFR/ERK signaling pathway in the behavioral responses to ethanol are unknown.

There is a need for methods of substance/drug abuse signal transduction, inhibition, and/or activation of signaling targets which can be used for the modulation of drug-induced impairment, such as, for example, sedation, locomotor impairment, or sensitivity.

### 4. SUMMARY OF THE INVENTION

The present invention is based in part on the discovery of a novel regulator of EGFR/ERK signaling, termed happyhour. While not intending to be bound by any particular theory of operation, the present invention is based, in part, on the identification and characterization of loss-of-function mutants in the happyhour (hppy) gene, which show a marked resistance to drug-induced impairment, for example, sedation and locomotory effects in Drosophila.

Accordingly, in a first aspect, the present invention provides an isolated happyhour polypeptide. In some embodiments, the polypeptide comprises an amino acid sequence having at least 95% identity to SEQ ID NO:1 OR 3. In some embodiments, the isolated polypeptide comprises the amino acid sequence of SEQ ID NO:1 OR 3.

In another aspect, the invention provides an isolated nucleic acid encoding a happyhour polypeptide. In some embodiments, the nucleic acid sequence encodes a polypeptide comprising an amino acid sequence having at least 95% identity to SEQ ID NO:1 OR 3. In certain embodiments, the isolated nucleic acid encodes a polypeptide comprising the amino acid sequence of SEQ ID NO:1 OR 3.

In certain embodiments, the isolated nucleic acid comprises a nucleic acid sequence having at least 95% identity to at least about 500 contiguous nucleotides selected from SEQ ID NO:2 OR 4 or the complement thereof. In certain embodiments, the isolated nucleic acid comprises at least about 500 nucleotides selected from the nucleic acid sequence of SEQ ID NO:2 OR 4, or the complement thereof. In a particular embodiment, the isolated nucleic acid comprises the nucleic acid sequence of SEQ ID NO:2 OR 4, or the complement thereof.

In another aspect, the invention provides an isolated oligonucleotide. In some embodiments, the oligonucleotide comprises at least about 10 consecutive nucleotides of SEQ ID NO:2 OR 4 or its complementary strand.

In another aspect, the invention provides a vector comprising an isolated nucleic acid of happyhour. In certain embodiments, the vector comprises at least about 500 nucleotides selected from the nucleic acid sequence of SEQ ID NO:2 OR 4. In certain embodiments, the vector comprises the nucleic acid sequence of SEQ ID NO:2 OR 4. In a particular embodiment, the happyhour nucleic acid sequence in the vector is operably linked to a transcriptional regulatory sequence. In certain embodiments, the vector is selected from the group comprising a plasmid, a cosmid, a virus, and a bacteriophage. In certain embodiments, the vector expresses a polypeptide comprising SEQ ID NO:1 OR 3 in a cell transformed with said vector.

In another aspect, the invention provides an isolated host cell comprising a happyhour nucleic acid according to the present invention. In another aspect, the invention provides an isolated host cell comprising a vector that expresses happyhour.

In another aspect, the invention provides an antibody that specifically binds to a happyhour polypeptide. In some embodiments, the antibody specifically binds to a happyhour polypeptide comprising an amino acid sequence of SEQ ID NO:1 OR 3. In certain embodiments, the antibody is a polyclonal, monoclonal, single chain monoclonal, recombinant, chimeric, humanized, mammalian, or human antibody.

In another aspect, the invention provides a method of screening for agents that are able to modulate happyhour activity, comprising: a) contacting a cell that expresses a happyhour polypeptide with a candidate agent; and b) assessing a biological activity of the happyhour. In certain embodiments, the biological activity is selected from the group consisting of modulation of ethanol sedation.

In another aspect, the invention provides a method of screening for agents that are able to modulate drug-induced impairment, comprising: a) contacting a cell with a candidate agent capable of modulating EGFR; b) assessing a biological activity of EGFR; and c) correlating the biological activity of EGFR with a biological activity of happyhour in the cell.

In another aspect, the invention provides a method of detecting the presence of the happyhour nucleic acid in a sample, comprising: (a) contacting the sample with a nucleic acid that hybridizes to the happyhour nucleic acid, and (b) determining whether the nucleic acid binds to a nucleic acid in the sample.

In another aspect, the invention provides a method for identifying whether a subject is genetically predisposed to increased ethanol sedation, comprising, detecting in a biological sample from the subject, a happyhour gene associated with increased ethanol sedation.

In another aspect, the invention provides a method for identifying whether a subject is genetically predisposed to decreased ethanol sedation, comprising, detecting in a biological sample from the subject, a happyhour gene associated with decreased ethanol sedation.

In another aspect, the invention provides a composition comprising a happyhour nucleic acid of the invention and a pharmaceutically acceptable carrier. In some embodiments, the composition comprises a polynucleotide encoding a polypeptide having an amino acid sequence that comprises SEQ ID NO: 1 OR 3 and a pharmaceutically acceptable carrier. In certain embodiments, the polynucleotide comprises a nucleotide sequence of SEQ ID NO:2 OR 4.

In another aspect, the invention provides a kit comprising i) an isolated oligonucleotide comprising at least 10 consecutive nucleotides of SEQ ID NO:2 OR 4, or its complementary strand; and ii) a container. In certain embodiments, the kit contains the oligonucleotide which comprises at least 15 consecutive nucleotides of SEQ ID NO:2 OR 4 or its complementary strand.

### 5. BRIEF DESCRIPTION OF THE DRAWINGS

FIG. 1. hppy mutants display increased resistance to ethanol induced sedation. (A) hppy¹⁷-51 flies show increased resistance to ethanol induced sedation and decreased locomotor impairment as measured by the locomotor tracking system. Here and elsewhere, locomotion and sedation were measured in sample populations of ˜25 flies. The average speeds of hppy¹⁷⁻⁵¹ flies and control line 8-165 (Ctl) flies were plotted as a function of time. Ethanol exposure (110 U ethanol vapor/40 U air; 110/40 E/A) commenced at 0 min and was continuous thereafter. hppy¹⁷⁻⁵¹ flies showed increased locomotor speed compared to the control lines at 15, 17.5 and 20 minutes. Individual time points analyzed by Student's unpaired t-tests assuming equal variance, with the critical p value adjusted to α=0.01, revealed significant differences at 20 minutes (p=0.0010), 17.5 minutes (p=0.0018), and 15 minutes (p=0.0007) (n=6). (B) hppy¹⁷⁻⁵¹ flies have normal ethanol absorption. Ethanol levels were measured in extracts of hppy¹⁷⁻⁵¹ and control flies (see Methods). Two way ANOVA analysis failed to reveal a significant difference between genotypes (p=0.1480) (n=4). (C, D) hppy¹⁷⁻⁵¹ flies show increased resistance to ethanol induced sedation as measured by the loss of righting (LOR) assay (C), while excision of the P-element in hppy¹⁷⁻⁵¹ (exc. 3 and 16) reverts the sedation resistance phenotype. Ethanol exposure (110/40 E/A) commenced at 0 min and was continuous thereafter. n=8. (D) The median sedation time (ST50)—the time required for half of the ethanol-exposed flies to show LOR—was calculated by linear interpolation (see Methods). One-way ANOVA followed by post-hoc Newman-Keuls testing revealed significant differences between the ST50 of hppy¹⁷⁻⁵¹ and control flies (p<0.001) whereas the ST50 values of the two excisions were not significantly different from that of the control line (p>0.05) (n=8). (E, F) hppy^{KG5537} flies, also showed increased resistance to ethanol induced sedation. (E) Sedation profiles and (F) ST50 values were calculated for hppy¹⁷⁻⁵¹, control, and hppy^{KG5537}. One-way ANOVA of the ST50 values followed by post-hoc Newman-Keuls tests revealed a significant difference between genotypes (p<0.0001), with both hppy insertions significantly more resistant to ethanol induced sedation than the control (p<0.001 for each comparison) (n=12). In this an all figures, error bars represent the standard error of the mean (SEM) and stars denote statistical significance (*p<0.05, **p<0.01, ***p<0.001).

FIG. 2. Molecular characterization of the hppy gene region (CG7097). (A) Cartoon of the hppy transcription unit. Exons are shown as boxes. M indicates the translation start site, while * indicates the translation stop codon. Blue arrows indicate the regions amplified for quantitative RT-PCR analysis. The structures of the two transcripts, hppy-RA and hppy-RB, are diagrammed, as well as the insertion sites of hppy¹⁷⁻⁵¹ and hppy^{KG5537}. (B) A schematic of the HPPY protein. Proteins encoded by hppy-RA (SEQ ID NO:1) and lippy-RB (SEQ ID NO:3) contain a serine/threonine kinase near the N-terminus and a citron homology domain near the C-terminus. (C, D) Expression of hppy is reduced in hppy mutants. RNA was isolated from whole flies and subjected to quantitative RT-PCR (QPCR). Analysis was also performed on mRNA isolated from heads and bodies separately and similar results were found (i.e., transcript reductions were comparable in heads and bodies, data not shown). Relative mRNA levels are expressed as fold increase over w Berlin (wB) control RNA. All QPCR experiments on whole fly mRNA were repeated with essentially identical results (data not shown). (C) QPCR on whole flies using a primer/probe set recognizing both hppy-RA and hppy-RB transcripts. One-way ANOVA revealed a significant difference between genotypes (p<0.0001). Post-hoc Newman-Keuls tests revealed a significant difference between hppy¹⁷⁻⁵¹ and both wB and Ctl (p<0.001) and between hppy^{KG5537} and both controls (p<0.001 for each comparison) (n=3). (D) QPCR on whole flies using a primer/probe set recognizing specific for the hppy-RA transcript. One-way ANOVA revealed a significant difference between genotypes (p<0.0001). Post-hoc Newman-Keuls tests revealed a significant differences between hppy¹⁷⁻⁵¹ and both controls (p<0.001) and between hppy^{KG5537} and both controls (p<0.001) (n=3).

FIG. 3. Phenotypic rescue and overexpression of lippy. (A, B) The hppy¹⁷⁻⁵¹ GAL4 expression pattern is widespread in the fly CNS. Pictured is an image of the adult brain (A) and ventral nerve cord (B) of a fly harboring hppy17-51 and UAS-GFP. (C) The hppy 17-51 sedation resistance phenotype can be rescued by expression of the UAS-hppy^{RB1} transgene in the hppy 17-51 homozygous mutant background. One-way ANOVA of the ST50 values revealed a significant difference between genotypes (p<0.0001). Post-hoc Newman-Keuls analysis revealed a significant difference between hppy17-51; UAS-hppy^{RB1} and hppy17-51 (p<0.001). ST50 of hppy¹⁷⁻⁵¹ UAS-hppy^{RB1} was not significantly different from Control (p)>0.05) or UAS-hppy^{RB1} (p>0.05) (n=8-12). (D) Introduction of the UAS-hppy^{RB1} transgene into the hppy^{KG5537} mutant background, which lacks GAL4 activity, does not rescue sedation resistance. One-way ANOVA of the ST50 values revealed a significant difference between genotypes (p<0.0001). Post-hoc Newman-Keuls analysis revealed a difference between hppy^{KG5537}; UAS-hppy^{RB1} and Control (p<0.001) as well as between hppy^{KG5537}; UAS-hppy^{RB1} and UAS-hppy^{RB1} (p<0.001). hppy^{KG5537} UAS-hppy^{RB1} was not significantly different from hppy^{KG5537} (p>0.05) (n=8). (E) Pan-neuronal expression of UAS-hppy^{RB1} under the control of the elav-GAL4^{c155} driver rescues the sedation resistance phenotype of hppy^{KG5537} flies. One-way ANOVA of the ST50 values revealed a significant difference between genotypes (p<0.0001). Post-hoc Newman-Keuls analysis revealed a difference between elav-GAL4^{c155}; hppy^{KG5537}/hppy^{KG5537}; UAS-hppy^{RB1}/+ and hppy^{KG5537}/hppy^{KG5537} (p<0.001), elav-GAL4^{c155}; hppy^{KG5537}/hppy^{KG5537} (p<0.001), and hppy^{KG5537}/hppy^{KG5537}; UAS-hppy^{RB1}/+ (p<0.001). ST50 of elav-GAL4^{c155}; hppy^{KG5537}/hppy^{KG5537}; UAS-hppy^{RB1}/+ was not significantly different from Control (p>0.05) (n=8). (F) Neuronal overexpression of UAS-hppy^{RB1} using the elav-GAL4^{c155} driver increases sensitivity to ethanol induced sedation. One-way ANOVA of ST50 values revealed a significant difference between genotypes (0<0.0001). Post-hoc Newman-Keuls analysis revealed a difference between elav-GAL4^{c155}; UAS-hppy^{RB1}/+ and elav-GAL4^{c155} (p<0.001) and between elav-GAL4^{c155}; UAS-hppy^{RB1}/+ and UAS-hppy^{RB1}/+ (p<0.001) (n=8).

FIG. 4. Perturbation of JNK signaling does not alter ethanol sensitivity as measured in the ethanol LOR assay. (A, B) Flies expressing a constitutively active JNKK, UAS-hep^{ACT}, under the control of the pan-neuronal driver elav-GAL4^{3El} have an ST50 similar to those of controls. (A) Sedation profiles and (B) ST50 values are shown. One-way ANOVA of the ST50 values failed to reveal a significant difference between genotypes (p=0.0618) (n=8). (C, D) Flies expressing wild-type JNK, UAS-Bsk^{WT}, under the control of the pan-neuronal driver elav-GAL4^{c155} have an ST50 similar to those of controls. (C) Sedation profiles and (D) ST50 values are shown. One-way ANOVA of the ST50 values failed to reveal a significant difference between genotypes (p=0.0955) (n=8). (E, F) Flies expressing a dominant negative form of the transcription factor dJun, UAS-Jun^{DN}, under the control of the pan-neuronal driver elav-GAL4^{c155} have an ST50 similar to those of controls. (E) Sedation profiles and (F) ST50 values are shown. One-way ANOVA of the ST50 values failed to reveal a significant difference between genotypes (p=0.2254) (n=8).

FIG. 5. Activation of EGFR/ERK signaling in neuronal tissues decreases ethanol sensitivity as measured in the ethanol LOR assay. (A) Flies expressing a secreted form of the EGFR ligand Spitz, UAS-spi^{SEC}, under the control of the pan-organismal driver Tub-GAL4 are resistant to ethanol-induced sedation. One-way ANOVA of the ST50 values revealed a significant difference between genotypes (p<0.0001). Post-hoc Newman-Keuls analysis revealed a significant difference between Tub-GAL4; UAS-spi^{SEC} and Tub-GAL4 (p<0.01) as well as between Tub-GAL4; UAS-spi^{SEC} and UAS-spi^{SEC} (<0.001) (n=8). (B) Flies expressing a wild-type form of the EGFR, UAS-egfr^{WT}, under the control of the pan-neuronal driver elav-GAL4^{c155} are resistant to ethanol-induced sedation. One-way ANOVA of the ST50 values revealed a significant difference between genotypes (p<0.0001). Post-hoc Newman-Keuls analysis revealed a significant difference between elav-GAL4^{c155}; UAS-egfr^{WT} and elav-GAL4^{c155} (p<0.001) as well as between elav-GAL4^{c155}; UAS-egfr^{WT} and UAS-egf^{WT} (p<0.001) (n=8). (C) Flies expressing a constitutively active form of the ERK rolled, UAS-rl^{ACT}, under the control of the pan-neuronal driver elav-GAL4^{c155} are resistant to ethanol-induced sedation. One-way ANOVA of the ST50 values revealed a significant difference between genotypes (p=0.0002). Post-hoc Newman-Keuls analysis revealed a significant difference between elav-GAL4^{c155}; UAS-rl^{ACT} and elav-GAL4^{c155} (p<0.01) as well as between elav-GAL4^{c155}; UAS-rl^{ACT} and UAS-rl^{ACT} (p<0.001) (n=7-8).

FIG. 6. Inhibition of EGFR/ERK signaling increases ethanol sensitivity as measured in the LOR assay. The P-element induced loss-of-function rhomboid-l mutant, rho-l^{A0544}, displays enhanced sensitivity to ethanol-induced sedation. (A, B) rho^{A0544} flies are sensitive to ethanol-induced sedation as measured in the LOR assay. (A) Sedation profiles and (B) ST50 values of rho^{A0544} flies and control flies are shown. Student's unpaired t-test assuming equal variance of the ST50 values revealed a significant difference between genotypes (p<0.0001) (n=8). (C) Expression of rhomboid-l is reduced in rho-l^{A0544} mutants. RNA was isolated from heads and QPCR was performed as described (see Methods). Relative mRNA levels are expressed as fold increase over the control (line 8-165) RNA. Student's unpaired t-test assuming equal variance revealed a significant difference between genotypes (Q=0.0102). Quantitative RT-PCR was repeated using a second set of primers recognizing a more 5′ region of the rhomboid-l gene with similar results (data not shown) (n=3). (D) The P element-induced putative loss-of-function Star mutant, 01624, displays enhanced sensitivity to ethanol-induced sedation. Student's unpaired t-test assuming equal variance of ST50 values revealed a significant difference between genotypes (p<0.0001) (n=8). (E) A diagram of the rho-l gene region. Exons are shown as boxes. The insertion site of the P-element A0544 is indicated. Blue arrows delineate the region amplified for QPCR analysis. (F) A diagram of the Star gene region. Exons are shown as boxes. The insertion site of the P-element d01624 is indicated.

FIG. 7. Genetic interactions between EGFR pathway and hppy in the fly eye. (A-H) Genetic interactions in the fly eye. GMR-GAL4-driven hppy-RB expression suppresses and enhances the rough eye phenotype caused by overexpression of EGFR and Yan, respectively; hppy overexpression does not affect the rough eye of flies expressing an activated rolled transgene. Scanning electron micrographs of adult eyes of the following genotypes: (A) GMR-GAL4, (B) GMR-GAL4; UAS-hppy^{RB1}, (C) GMR-GAL4; UAS-egf^{WT}, arrow points to blister, (D) GMR-GAL4; UAS-egfr^{WT}; UAS-hppy^{RB1}, (E) GMR-GAL4; UAS-rl^{ACT}, (F) GMR-GAL4; UAS-rl^{ACT}; UAS-hppy^{RB1}, (C) GMR-GAL4; UAS-yan. (H) GMR-GAL4; UAS-yan, UAS-hppy^{RB1}. Flies are heterozygous for all transgenes. Anterior is to the right, dorsal is up.

FIG. 8. QPCR analysis of UAS-hppy^{RB} transgene expression in hppy17-51 flies and rescue by the UAS-hppy^{RB2} transgene. (A) QPCR on whole-fly mRNA using a primer/probe set recognizing both hppy-RA and hppy-RB transcripts shows that UAS-hppy expression is increased in hppy¹⁷⁻⁵¹; UAS-hppy^{RB} flies. UAS hppy^{RB1} and UAS-hppy^{RB2} represent two different insertions lines of the UAS-hppy^{RB} transgene. All behavioral experiments presented in the main body of the paper utilized the more strongly expressing, UAS-hppy^{RB} insertion line. One-way ANOVA revealed a significant difference between genotypes (p<0.001). Post-hoc Newman-Keuls tests revealed a significant difference between hppy¹⁷⁻⁵¹; UAS-hppy^{RB1} and both w Berlin (p<0.001) and Control (line 8-165) (p<0.001) as well as a significant difference between hppy¹⁷⁻⁵¹, UAS-hppy^{RB2} and both w Berlin (p<0.001) and Control (p<0.001) (n=3). (B) QPCR on whole-fly mRNA using a primer/probe set recognizing specifically the hppy-RA transcript shows that the hppy-RA transcript is not increased in hppy¹⁷⁻⁵¹; UAS-hppy^{RB} flies. One-way ANOVA revealed a significant difference between genotypes (p<0.001). Post-hoc Newman-Keuls tests revealed a significant difference between hppy¹⁷⁻⁵¹; UAS-hppy^{RB1} and both w Berlin (p<0.001) and Control line 8-165 (p<0.001) as well as a significant difference between hppy¹⁷⁻⁵¹; UAS-hppy^{RB2} and both w Berlin (p<0.001) and Control (p<0.001) (n=3). (C, D) The hppy¹⁷⁵ sedation resistance phenotype can be partially rescued by expression of the UAS-hppy^{RB2} transgene in the hppy¹⁷⁻⁵¹ mutant background. (C) Sedation profiles and (D) ST50 values are shown. One-way ANOVA of ST50 values revealed a significant difference between genotypes (p<0.0001). Post-hoc Newman-Keuls tests revealed a significant difference between hppy¹⁷⁻⁵¹; UAS-hppy^{RB2} and hppy¹⁷⁻⁵¹ (p<0.001). hppy¹⁷⁻⁵¹; UAS-hppy^{RB2} was also significantly different from Control (p<0.001) and UAS-hppy^{RB2} (p<0.001) (n=8).

FIG. 9. hppy¹⁷⁻⁵¹ flies are resistant to sedation when exposed to a broad range of ethanol concentrations. Sedation profiles were calculated for hppy¹⁷⁻⁵¹ flies and the control line 8-165 (Ctl) flies at a variety of ethanol concentrations. Ethanol exposure commenced at 0 min and was continuous thereafter. Ethanol/air (E/A) concentrations tested were: (A) 70/80 E/A, (B) 90/60 E/A, (C) 130/20 E/A, and (D) 150/0 E/A (n=8).

FIG. 10. hppy¹⁷⁻⁵¹ flies do not have defects in negative geotaxis. The ability of hppy17-51 and control (line 8-165) flies to climb to the top of a glass cylinder after being banged down to the bottom was measured. Student's paired t-test assuming equal variances failed to reveal a significant difference between genotypes (p=0.4852) (n=25).

FIG. 11. hppy expression levels during development and adulthood. hppy expression levels in Control and hppy¹⁷⁻⁵¹ flies were assayed by QPCR during different stages of development, using a primer/probe set recognizing both hppy-RA and hppy-RB transcripts. Developmental stages are: embryos (Day 1), 2^{nd} instar larvae (Day 3), 3^{rd} instar wandering larvae (Day 5), and pupae (Day 7), and data was compared to hppy levels in Control adult flies (Days 12-14) (n=3).

FIG. 12 presents the study on the p38 pathway in comparison with study on the JNK pathway. (A) Flies expressing a constitutively active JNKK, UAS-hep^{ACT}, under the control of the pan-neuronal driver elav-GAL4^{3El} have an ST50 similar to those of controls. One-way ANOVA of ST50 values failed to reveal a significant difference between genotypes (p=0.0618) (n=8). (B) Flies expressing wild-type JNK, UAS-bsk^{WT}, under the control of the pan-neuronial driver elav-GAL4^{c155} have an ST50 similar to those of controls. One-way ANOVA of ST50 values failed to reveal a significant difference between genotypes (p=0.0955) (n=8). (C) Flies expressing a dominant negative form of the transcription factor dJun, UAS-jun^{DN}, under the control of the pan-neuronal driver elav-GAL4^{c155} have an ST50 similar to those of controls. One-way ANOVA of ST50 values failed to reveal a significant difference between genotypes (p=0.2254). n=8. (D) Flies expressing the wild-type transcription factor dJun, UAS-Jun^{WT}, under the control of the pan-neuronal driver elav-GAL4^{c155} have an ST50 similar to those of controls. One-way ANOVA of ST50 values failed to reveal a significant difference between genotypes (p=0.0611) (n=8). (E) Flies expressing a dominant negative form of the MAPK p380, UAS-p38b^{DN}, under the control of the pan-organismal driver Tub-GAL4 have an ST50 similar to those of controls. One-way ANOVA of ST50 values failed to reveal a significant difference between genotypes (p=0.8513) (n=4).

### 6. DETAILED DESCRIPTION OF THE INVENTION

This disclosure provides an isolated happyhour-encoding nucleic acids, isolated happyhour polypeptides, oligonucleotides that hybridize to happyhour nucleic acids, and expression vectors containing happyhour polypeptide-encoding sequences. The present invention further provides isolated host cells, antibodies, compositions, and kits relating to happyhour. In other aspects, the present invention further provides methods of screening for agents that modulate happyhour activity. The agents are useful, for example for modulating ethanol sensitivity when administered to a subject in need thereof. Happyhour is believed to be linked to, inter alia, modulation of ethanol sedation, locomotor impairment in Drosophila.

Having herein provided the nucleotide sequence of the happyhour cDNA, correspondingly provided are the complementary DNA strands of the cDNA molecule, and DNA molecules which hybridize under stringent conditions to happyhour cDNA molecule, or its complementary strand. Such hybridizing molecules include DNA molecules differing only by minor sequence changes, including nucleotide substitutions, deletions and additions. Also comprehended by this invention are isolated oligonucleotides comprising at least a portion of the cDNA molecule or its complementary strand. These oligonucleotides can be employed as effective DNA hybridization probes or primers for use in the polymerase chain reaction.

Recombinant DNA vector comprising the disclosed DNA molecules, and transgenic host cells containing such recombinant vectors, are also provided. Disclosed embodiments also include transgenic nonhuman animals which over- or under-express happyhour protein, or over- or under-express fragments or variants of happyhour protein.

For clarity of disclosure, and not by way of limitation, the detailed description of the invention hereinafter is divided into the subsections that follow. All publications mentioned herein are incorporated by reference to disclose and describe the methods and/or materials in connection with which the publications are cited.

### 6.1 Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” mean “at least one” or “one or more” unless the context clearly dictates otherwise.

As used herein, “happyhour” refers to proteins or peptides which have an amino acid sequence that is identical to SEQ ID NO:1 OR 3, as well as proteins sharing sequence similarity, e.g., 70%, 75%, 80%, 85%, 90%, 95%, or greater percent identity, with the amino acid sequence of SEQ ID NO:1 OR 3. Further, these proteins can have a biological activity in common with the polypeptide having the amino acid sequence of SEQ ID NO:1 OR 3, including, but not limited to, antigenic cross-reactivity, autoinhibition, phosphorylation activity, and the like. It is also contemplated that a happyhour protein can have one or more conservative or non-conservative amino acid substitutions, or additions or deletions from the amino acid sequence of SEQ ID NO:1 OR 3 so long as the protein having such sequence alteration shares a biological activity as described above with the polypeptide of SEQ ID NO:1 OR 3. Happyhour also includes proteins or peptides expressed from different mutations, different spliced forms and various sequence polymorphisms of the happyhour gene.

As used herein. “functional fragments and variants of” “happyhour” refer to those fragments and variants that maintain one or more functions of happyhour. It is recognized that the gene or cDNA encoding happyhour can be considerably mutated without materially altering one or more happyhour functions. First, the genetic code is well-known to be degenerate, and thus different codons may encode the same amino acids. Second, even where an amino acid substitution is introduced, the mutation can be conservative and have no material impact on the essential functions of happyhour. Third, part of the happyhour polypeptide can be deleted without impairing or eliminating all of its functions. Fourth, insertions or additions can be made in happyhour, for example, adding epitope tags, without impairing or eliminating its functions. Other modifications can be made without materially impairing one or more functions of happyhour, for example, in vivo or in vitro chemical and biochemical modifications which incorporate unusual amino acids. Such modifications include, for example, acetylation, carboxylation, phosphorylation, glycosylation, ubiquination, labeling with radionuclides, and various enzymatic modifications, as will be readily appreciated by those skilled in the art. A variety of methods for labeling proteins and substituents or labels useful for such purposes are well known in the art, and include radioactive isotopes such as ligands which bind to labeled antiligands (e.g., antibodies), fluorophores, chemiluminescent agents, enzymes, and antiligands. Functional fragments and variants can be of varying length. For example, some fragments have at least 10, 25, 50, 75, 100, or 200 or more amino acid residues.

As used herein, “protein” is synonymous with “polypeptide” or “peptide” unless the context clearly dictates otherwise.

As used herein, a “happyhour gene” refers to a gene that encodes happyhour as defined herein. A mutation of happyhour gene includes nucleotide sequence changes, additions or deletions, including deletion of large portions or the entire happyhour gene, or duplications of all or substantially all of the gene. Alternatively, genetic expression of happyhour can be deregulated such that happyhour is over or under expressed. The term “happyhour gene” is understood to include the various sequence polymorphisms and allelic variations that exist within the population. This term relates primarily to an isolated coding sequence, but can also include some or all of the flanking regulatory elements and/or intron sequences. The RNA transcribed from a mutant happyhour gene is mutant happyhour messenger RNA.

As used herein, “happyhour cDNA” refers to a cDNA molecule which, when transfected or otherwise introduced into cells, expresses the happyhour protein. The happyhour cDNA can be derived, for instance, by reverse transcription from the mRNA encoded by the happyhour gene and lacks internal non-coding segments and transcription regulatory sequences present in the happyhour gene. An exemplary human happyhour cDNA is shown as SEQ ID NO:2 or 4.

As used herein, “vector” refers to discrete elements that are used to introduce heterologous DNA into cells for either expression or replication thereof. Selection and use of such vehicles are well known within the skill of the artisan. An expression vector includes vectors capable of expressing DNA that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “biological sample” includes tissues, cells and biological fluids isolated from a subject, as well as tissues, cells and fluids present within a subject.

As used herein, the term “pharmaceutically acceptable” means approved by a regulatory agency of the Federal or a state government, or listed in the U.S. Pharmacopeia or other generally recognized pharmacopeia for use in animals, and more particularly in humans.

As used herein, the term “carrier” refers to a diluent, adjuvant, excipient, or vehicle with which a therapeutic of the invention is administered. Such pharmaceutical carriers can be sterile liquids, such as water and oils, including those of petroleum, animal, vegetable or synthetic origin, such as peanut oil, soybean oil, mineral oil, sesame oil and the like.

As used herein, an “effective amount” of an active agent for treating a particular disease is an amount that is sufficient to ameliorate, or in some manner reduce the symptoms associated with the disease. The amount may cure the disease but, typically, is administered in order to ameliorate the symptoms of the disease.

As used herein, “active agent” means any substance intended for the diagnosis, cure, mitigation, treatment, or prevention of disease in humans and other animals, or to otherwise enhance physical and mental well being.

### 6.2 Polypeptides of the Invention

The present invention provides newly identified and isolated polypeptides referred to in the present application as happyhour. In some embodiments, the polypeptides comprise substantially the same amino acid sequences as found in the native happyhour sequences. In certain embodiments, the invention provides amino acid sequences of functional fragments and variants of happyhour that comprise an antigenic determinant (i.e., a portion of a polypeptide that can be recognized by an antibody) or which are otherwise functionally active, as well as nucleic acids encoding the foregoing. Happyhour functional activity encompasses one or more known functional activities associated with a modulation of ethanol sedation; antigenicity (the ability to be bound by an antibody to a protein consisting of the amino acid sequence of SEQ ID NO: 1 OR 3); immunogenicity (the ability to induce the production of an antibody that binds SEQ ID NO: 1 OR 3), and so forth.

In some embodiments, the polypeptides comprise the amino acid sequences having functionally inconsequential amino acid substitutions, and thus have amino acid sequences which differ from that of the native happyhour sequence. Substitutions can be introduced by mutation into happyhour-encoding nucleic acid sequences that result in alterations in the amino acid sequences of the encoded happyhour but do not alter happyhour function. For example, nucleotide substitutions leading to amino acid substitutions at “non-essential” amino acid residues can be made in happyhour encoding sequences. A “non-essential” amino acid residue is a residue that can be altered from the wild-type sequence of happyhour without altering myosin light chain kinase biological activity, whereas an “essential” amino acid residue is required for such biological activity. For example, amino acid residues that are conserved among the happyhour polypeptides of the invention are predicted to be particularly unsuitable for alteration. Amino acids for which conservative substitutions can be made are well known in the art.

Useful conservative substitutions are shown in Table 1, “Preferred Substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table 2 as exemplary are introduced and the products screened for happyhour polypeptide biological activity.

TABLE 1
Preferred Substitutions

| | | |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that effect: (1) the structure of the polypeptide backbone, such as a β-sheet or α-helical conformation; (2) the charge; (3) hydrophobicity; or (4) the bulk of the side chain of the target site, can modify happyhour polypeptide function or immunological identity. Residues are divided into groups based on common side-chain properties as denoted in Table 2. Non-conservative substitutions entail exchanging a member of one of these classes for another class. Substitutions may be introduced into conservative substitution sites or more preferably into non-conserved sites.

TABLE 2
Amino acid classes

| Class | Amino Acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (see Carter, Biochem. J 237:1-7 (1986); Zoller and Smith, Methods Enzymol. 154:329-50 (1987)), cassette mutagenesis, restriction selection mutagenesis (Wells et al., Gene 34:315-323 (1985)) or other known techniques can be performed on cloned happyhour-encoding DNA to produce happyhour variant DNA (Ausubel et al., Current Protocols In Molecular Biology, John Wiley and Sons, New York (current edition); Sambrook et al., Molecular Cloning A Laboratory Manual 3d. ed., Cold Spring Harbor Laboratory Press, Cold Spring Harbor, N.Y. (2001).

In certain embodiments, happyhour used in the present invention includes happyhour mutants or derivatives having an amino acid substitution with a non-classical amino acid or chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, O-alanine, fluoro-amino acids, designer amino acids such as β-methyl amino acids, Cα-methyl amino acids, Nα-methyl amino acids, and amino acid analogs in general.

In one embodiment, the present invention includes an isolated polypeptide comprising an amino acid sequence having at least 70% identity to SEQ ID NO:1 OR 3. In some embodiments, the polypeptide comprises an amino acid sequence having at least 75%, 80%, 85%, 90%, or 95% identity to SEQ ID NO:1 OR 3. In a particular embodiment, the isolated polypeptide comprises the amino acid sequence of SEQ ID NO:1 OR 3.

The present invention also includes an isolated polynucleotide comprising an nucleic acid sequence having at least 70% identity to SEQ ID NO:2 OR 4. In some embodiments, the polynucleotide comprises an nucleic acid sequence having at least 75%, 80%, 85%, 90%, or 95% identity to SEQ ID NO:2 OR 4. In a particular embodiment, the isolated polynucleotide comprises the nucleic acid sequence of SEQ ID NO:2 OR 4.

Percent identity in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology. In certain embodiments, a happyhour homologue is characterized by its percent sequence identity or percent sequence similarity with the naturally occurring happyhour sequence. Sequence homology, including percentages of sequence identity and similarity, are determined using sequence alignment techniques well-known in the art, preferably computer algorithms designed for this purpose, using the default parameters of said computer algorithms or the software packages containing them.

Non-limiting examples of computer algorithms and software packages incorporating such algorithms include the following. The BLAST family of programs exemplify a preferred, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another preferred example is the algorithm of Myers and Miller (1988 CABIOS 4:11-17) which is incorporated into the ALIGN program (version 2.0) and is available as part of the GCG sequence alignment software package. Also preferred is the FASTA program (Pearson W. R. and Lipman D. J., Proc. Slat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions. Additional methods, e.g., protein affinity chromatography, affinity blotting, in vitro binding assays, and the like, will be apparent to skilled artisans apprised of the present invention.

For the purpose of comparing two different nucleic acid or polypeptide sequences, one sequence (test sequence) may be described to be a specific “percent identical to” another sequence (reference sequence) in the present disclosure. In this respect, when the length of the test sequence is less than 90% of the length of the reference sequence, the percentage identity is determined by the algorithm of Myers and Miller, Bull. Math. Biol., 51:5-37 (1989) and Myers and Miller, Comput. Appl. Biosci., 4(1):11-17 (1988). Specifically, the identity is determined by the ALIGN program. The default parameters can be used.

Where the length of the test sequence is at least 90% of the length of the reference sequence, the percentage identity is determined by the algorithm of Karlin and Altschul, Proc. Natl. Acad. Sci. USA, 90:5873-77 (1993), which is incorporated into various BLAST programs. Specifically, the percentage identity is determined by the “BLAST 2 Sequences” tool. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff-50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter).

In certain embodiments, the polypeptides of the present invention are capable of modulating ethanol sedation in Drosophila as described in the examples below. In certain embodiments, the polypeptides of the invention are useful for screening for agents capable of modulating ethanol sedation in a subject in need thereof.

### 6.3 Nucleic Acids of the Invention

In another aspect, the present invention provides newly identified and isolated nucleotide sequences encoding rat happyhour and human happyhour respectively. In particular, nucleic acids encoding native sequence rat happyhour and native sequence human happyhour polypeptides have been identified and isolated.

The happyhour-encoding or related sequences provided by the instant invention include those nucleotide sequences encoding substantially the same amino acid sequences as found in native happyhour, as well as those encoded amino acid sequences having functionally inconsequential amino acid substitutions, and thus have amino acid sequences which differ from that of the native sequence. Examples include the substitution of one basic residue for another (i.e. Arg for Lys), the substitution of one hydrophobic residue for another (i.e. Leu for Ile), or the substitution of one aromatic residue for another (i.e. Phe for Tyr, etc.).

The invention further relates to fragments of happyhour. Nucleic acids encoding such fragments are thus also within the scope of the invention. The happyhour gene and happyhour-encoding nucleic acid sequences of the invention include human and related genes (homologues) in other species. In some embodiments, the happyhour gene and happyhour-encoding nucleic acid sequences are from vertebrates, or more particularly, mammals. In some embodiments, the happyhour gene and happyhour-encoding nucleic acid sequences are of rat origin. In a preferred embodiment of the invention, the happyhour gene and happyhour-encoding nucleic acid sequences are of human origin.

In one aspect, the invention provides an isolated nucleic acid encoding a polypeptide comprising an amino acid sequence having at least 70% identity to SEQ ID NO:1 OR 3. In some embodiments, the nucleic acid encodes a polypeptide comprising an amino acid sequence having at least 75%, 80%, 85%, 90%, or 95% identity to SEQ ID NO:1 OR 3. In a particular embodiment, the isolated nucleic acid encodes a polypeptide comprising the amino acid sequence of SEQ ID NO:1 OR 3.

In another embodiment, the invention provides an isolated nucleic acid comprising a nucleic acid sequence having at least 70% identity to at least about 500 contiguous nucleotides selected from SEQ ID NO:2 OR 4 or the complement thereof. In some embodiments, the nucleic acid comprises a nucleic acid sequence having at least 70% identity to at least about 500, 600, 700, 800, 900, 1000, 1100, 1200, 1500, 2000, or 2500 contiguous nucleotides selected from SEQ ID NO: 2 OR 4. In some embodiments, the nucleic acid comprises a nucleic acid sequence having at least 75% identity to at least about 500 contiguous nucleotides selected from SEQ ID NO: 2 OR 4. In some embodiments, the nucleic acid comprises a nucleic acid sequence having at least 75% identity to at least about 500, 600, 700, 800, 900, 1000, 1100, 1200, 1500, 2000, or 2500 contiguous nucleotides selected from SEQ ID NO: 2 OR 4. In some embodiments, the nucleic acid comprises a nucleic acid sequence having at least 80% identity to at least about 500 contiguous nucleotides selected from SEQ ID NO: 2 OR 4. In some embodiments, the nucleic acid comprises a nucleic acid sequence having at least 80% identity to at least about 500, 600, 700, 800, 900, 1000, 1100, 1200, 1500, 2000, or 2500 contiguous nucleotides selected from SEQ ID NO: 2 OR 4. In some embodiments, the nucleic acid comprises a nucleic acid sequence having at least 85% identity to at least about 500 contiguous nucleotides selected from SEQ ID NO: 2 OR 4. In some embodiments, the nucleic acid comprises a nucleic acid sequence having at least 85% identity to at least about 500, 600, 700, 800, 900, 1000, 1100, 1200, 1500, 2000, or 2500 contiguous nucleotides selected from SEQ ID NO: 2 OR 4. In some embodiments, the nucleic acid comprises a nucleic acid sequence having at least 90% identity to at least about 500 contiguous nucleotides selected from SEQ ID NO: 2 OR 4. In some embodiments, the nucleic acid comprises a nucleic acid sequence having at least 90% identity to at least about 500, 600, 700, 800, 900, 1000, 1100, 1200, 1500, 2000, or 2500 contiguous nucleotides selected from SEQ ID NO: 2 OR 4. In some embodiments, the nucleic acid comprises a nucleic acid sequence having at least 95% identity to at least about 500 contiguous nucleotides selected from SEQ ID NO: 2 OR 4. In some embodiments, the nucleic acid comprises a nucleic acid sequence having at least 95% identity to at least about 500, 600, 700, 800, 900, 1000, 1100, 1200, 1500, 2000, or 2500 contiguous nucleotides selected from SEQ ID NO: 2 OR 4. In certain embodiments, the isolated nucleic acid comprises at least about 500 nucleotides selected from the nucleic acid sequence of SEQ ID NO: 2 OR 4, or the complement thereof. In certain embodiments, the isolated nucleic acid comprises at least about 500, 600, 700, 800, 900, 1000, 1100, 1200, 1500, 2000, or 2500 nucleotides selected from the nucleic acid sequence of SEQ ID NO: 2 OR 4, or the complement thereof. In a particular embodiment, the isolated nucleic acid comprises the nucleic acid sequence of SEQ ID NO: 2 OR 4, or the complement thereof.

The present invention also includes nucleic acids that hybridize to or are complementary to the foregoing sequences. In specific aspects, nucleic acids are provided which comprise a sequence complementary to at least 20, 30, 40, 50, 100, 200 nucleotides or the entire coding region of happyhour, or the reverse complement (antisense) of any of these sequences. In a specific embodiment, a nucleic acid which hybridizes to a happyhour nucleic acid sequence (e.g., having part or the whole of sequence SEQ ID NO:2 or SEQ ID NO:4, or the complements thereof), under conditions of low stringency is provided.

By way of example and not limitation, procedures using such conditions of low stringency are as follows (see also Shilo and Weinberg, 1981, Proc. Natl. Acad. Sci. U.S.A. 78:6789-6792). Filters containing DNA can be pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations can be carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe can be used. Filters can be incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution can then be replaced with fresh solution and incubated an additional 1.5 h at 60° C. Filters may be blotted dry and exposed for autoradiography. If necessary, filters may be washed for a third time at 65-68° C. and re-exposed to film. Other conditions of low stringency which may be used are well known in the art (e.g., as employed for cross-species hybridizations).

In another specific embodiment, a nucleic acid that hybridizes to a nucleic acid encoding happyhour, or its reverse complement, under conditions of high stringency is provided. By way of example and not limitation, procedures using such conditions of high stringency are as follows. Prehybridization of filters containing DNA may be carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters may be hybridized for 48 h at 65° C. in prehybridization mixture containing 100 μg/ml denatured salmon sperm DNA and 5-20×10⁶ cpm of ³²P-labeled probe. Washing of tilters may be done at 37° C. for 1 h in a solution containing 2×SSC, 0.01% PVP, 0.01% Ficoll, and 0.01% BSA. This can be followed by a wash in 0.1×SSC at 50° C. for 45 minutes before autoradiography. Other conditions of high stringency that may be used are well known in the art.

### 6.3.1 Cloning of the Happyhour Gene or cDNA

The present invention further provides methods and compositions relating to the cloning of a gene or cDNA encoding happyhour. In one embodiment of the invention, expression cloning (a technique commonly known in the art), may be used to isolate a gene or cDNA encoding happyhour. An expression library may be constructed by any method known in the art. In one embodiment, mRNA (e.g., human) is isolated, and cDNA is made and ligated into an expression vector such that the cDNA is capable of being expressed by the host cell into which it is introduced. Various screening assays can then be used to select for the expressed happyhour product. In one embodiment, anti-happyhour antibodies can be used for selection.

In another embodiment of the invention, polymerase chain reaction (PCR) may be used to amplify desired nucleic acid sequences of the present invention from a genomic or cDNA library. Isolated oligonucleotide primers representing known happyhour-encoding sequences can be used as primers in PCR. In certain embodiments, the isolated oligonucleotide primer comprises at least 10 consecutive nucleotides of SEQ ID NO:2 or 4 or its complimentary strand. In a preferred aspect, the oligonucleotide primers represent at least part of the conserved segments of strong homology between happyhour-encoding genes of different species. The synthetic oligonucleotides may be utilized as primers to amplify by PCR sequences from RNA or DNA, preferably a cDNA library, of potential interest. Alternatively, one can synthesize degenerate primers for use in the PCR reactions.

In the PCR reactions, the nucleic acid being amplified can include RNA or DNA, for example, mRNA, cDNA or genomic DNA from any eukaryotic species. PCR can be carried out, e.g., by use of a Perkin-Elmer Cetus thermal cycler and Taq polymerase. It is also possible to vary the stringency of hybridization conditions used in priming the PCR reactions, to allow for greater or lesser degrees of nucleotide sequence similarity between a known happyhour nucleotide sequence and a nucleic acid homologue being isolated. For cross-species hybridization, low stringency conditions are preferred. For same-species hybridization, moderately stringent conditions are preferred. After successful amplification of a segment of a happyhour homologue, that segment may be cloned, sequenced, and utilized as a probe to isolate a complete cDNA or genomic clone. This, in turn, will permit the determination of the gene's complete nucleotide sequence, the analysis of its expression, and the production of its protein product for functional analysis. In this fashion, additional nucleotide sequences encoding happyhour or happyhour homologues may be identified.

The above recited methods are not meant to limit the following general description of methods by which clones of genes encoding happyhour or homologues thereof may be obtained.

Any eukaryotic cell potentially can serve as the nucleic acid source for the molecular cloning of the happyhour gene, happyhour cDNA or a homologue thereof. The nucleic acid sequences encoding happyhour can be isolated from vertebrate, mammalian, human, porcine, bovine, feline, avian, equine, canine, as well as additional primate sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell, or by PCR amplification and cloning. See, for example, Sambrook et al., Molecular Cloning. A Laboratory Manual, 3d. ed., Cold Spring Harbor Laboratory Press, Cold Spring Harbor, N.Y. (2001); Glover, D. M. (ed.), DNA Cloning: A Practical Approach, 2d. ed., MRL Press, Ltd., Oxford, U.K. (1995). Clones derived from genomic DNA may contain regulatory and intron DNA regions in addition to coding regions; clones derived from cDNA will contain only exon sequences. Whatever the source, the gene may be cloned into a suitable vector for propagation of the gene.

In the cloning of the gene from genomic DNA, DNA fragments are generated, some of which will encode the desired gene. The DNA may be cleaved at specific sites using various restriction enzymes. Alternatively, one may use DNase in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the desired gene may be accomplished in a number of ways. For example, if a happyhour gene (of any species) or its specific RNA is available and can be purified and labeled, the generated DNA fragments may be screened by nucleic acid hybridization to the labeled probe (Benton and Davis, Science 196:180 (1977); Grunstein and Hogness, Proc. Natl. Acad. Sci. U.S.A. 72:3961 (1975). Those DNA fragments with substantial homology to the probe will hybridize. It is also possible to identify the appropriate fragment by restriction enzyme digestion(s) and comparison of fragment sizes with those expected according to a known restriction map if such is available. Further selection can be carried out on the basis of the properties of the gene.

Alternatively, the presence of the gene may be detected by assays based on the physical, chemical, or immunological properties of its expressed product. For example, cDNA clones, or DNA clones that hybrid-select the proper mRNAs, can be selected that produce a protein having e.g., similar or identical electrophoretic migration, isoelectric focusing behavior, proteolytic digestion maps, substrate binding activity, or antigenic properties as known for a specific happyhour. If an antibody to a particular happyhour is available, that happyhour may be identified by binding of labeled antibody to the clone(s) putatively producing the happyhour in an ELISA (enzyme-linked immunosorbent assay)-type procedure.

A happyhour or homologue thereof can also be identified by mRNA selection by nucleic acid hybridization followed by in vitro translation. In this procedure, fragments are used to isolate complementary mRNAs by hybridization. Such DNA fragments may represent available, purified DNA of another species containing a gene encoding happyhour. Immunoprecipitation analysis or functional assays of the in vitro translation products of the isolated mRNAs identifies the mRNA and, therefore, the complementary DNA fragments that contain the desired sequences. In addition, specific mRNAs may be selected by adsorption of polysomes isolated from cells to immobilized antibodies specifically directed against a specific happyhour. A radiolabelled happyhour-encoding cDNA can be synthesized using the selected mRNA (from the adsorbed polysomes) as a template. The radiolabelled mRNA or cDNA may then be used as a probe to identify the happyhour-encoding DNA fragments from among other genomic DNA fragments.

Alternatives to isolating the happyhour genomic DNA include, but are not limited to, chemically synthesizing the gene sequence itself from a known sequence or making cDNA to the mRNA which encodes happyhour. For example RNA for the cloning of happyhour cDNA can be isolated from cells that express a happyhour gene. Other methods are possible and within the scope of the invention.

The identified and isolated happyhour or happyhour analog-encoding gene can then be inserted into an appropriate cloning vector. A large number of vector-host systems known in the art may be used. Possible cloning vectors include, but are not limited to, plasmids or modified viruses, but the vector system must be compatible with the host cell used. Such vectors include, but are not limited to bacteriophages such as lambda derivatives, or plasmids such as pBR322, pUC plasmid derivatives, or the pBluescript vector. (Stratagene). The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini. These ligated linkers may comprise specific chemically synthesized oligonucleotides encoding restriction endonuclease recognition sequences. In an alternative method, the cleaved vector and happyhour-encoding gene or nucleic acid sequence may be modified by homopolymeric tailing. Recombinant molecules can be introduced into host cells via transformation, transfection, infection, electroporation, etc., so that many copies of the gene sequence are generated.

In an alternative method, the desired gene may be identified and isolated after insertion into a suitable cloning vector in a “shotgun” approach. Enrichment for the desired gene, for example, by size fractionization, can be done before insertion into the cloning vector.

To generate multiple copies of the isolated happyhour-encoding gene, cDNA, or synthesized DNA sequence, host cells, for example competent strains of E. Coli, may be transformed with recombinant DNA molecules incorporating said sequences according to any technique known in the art. Thus, the gene may be obtained in large quantities by growing transformants, isolating the recombinant DNA molecules from the transformants and, when necessary, retrieving the inserted gene from the isolated recombinant DNA.

### 6.3.2 Expression Vectors

In still another aspect, the invention provides expression vectors for expressing isolated happyhour-encoding cDNA sequences. Generally, expression vectors are recombinant polynucleotide molecules comprising expression control sequences operatively linked to a nucleotide sequence encoding a polypeptide. Expression vectors can readily be adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring harbor, NY, and Ausubel et al., eds., Current Edition, Current Protocols in Molecular Biology, Greene Publishing Associates and Wiley Interscience, NY.

Useful promoters for use in expression vectors include, but are not limited to, a metallothionein promoter (e.g. the Drosophila metallothionein promoter), a constitutive adenovin's major late promoter, a dexamethasone-inducible MMTV promoter, a SV40 promoter, a MRP pol III promoter, a constitutive MPSV promoter, an RSV promoter, a tetracycline-inducible CMV promoter (such as the human immediate-early CMV promoter), and a constitutive CMV promoter.

The expression vectors should contain expression and replication signals compatible with the cell in which the happyhour-encoding sequences are to be expressed. Expression vectors useful for expressing happyhour-encoding sequences include viral vectors such as retroviruses, adenoviruses and adenoassociated viruses, plasmid vectors, cosmids, and the like. Viral and plasmid vectors are preferred for transfecting the expression vectors into mammalian cells. For example, the expression vector pcDNA1 (Invitrogen, San Diego, Calif.), in which the expression control sequence comprises the CMV promoter, provides good rates of transfection and expression into such cells.

The expression vectors can be introduced into the cell for expression of the happyhour-encoding sequence by any method known to one of skill in the art without limitation. Such methods include, but are not limited to, e.g., direct uptake of the recombinant DNA molecule by a cell from solution; facilitated uptake through lipofection using, e.g., liposomes or immunoliposomes; particle-mediated transfection; etc. See, e.g., U.S. Pat. No. 5,272,065; Goeddel et al., Methods in Enzymology, vol. 185, Academic Press, Inc., CA (1990); Krieger, Gene Transfer and Expression—A Laboratory Manual, Stockton Press, New York (1990); Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001).

The expression vectors can also contain a purification moiety that simplifies isolation of the expressed protein. For example, a polyhistidine moiety of. e.g., six histidine residues, can be incorporated at the amino terminal end of the protein. The polyhistidine moiety allows convenient isolation of the protein in a single step by nickel-chelate chromatography. In certain embodiments, the purification moiety can be cleaved from the remainder of the delivery construct following purification. In other embodiments, the moiety does not interfere with the function of the functional domains of the expressed protein and thus need not be cleaved.

### 6.3.3 Cells

In yet another aspect, the invention provides a cell comprising an expression vector for expression of happyhour polypeptides of the invention, or portions thereof. The cell is preferably selected for its ability to express high concentrations of the happyhour polypeptide to facilitate subsequent purification of the polypeptide. In certain embodiments, the cell is a prokaryotic cell, for example, E. coli. In a preferred embodiment, the happyhour polypeptide is properly folded and comprises the appropriate disulfide linkages when expressed in E. coli.

In other embodiments, the cell is a eukaryotic cell. Useful eukaryotic cells include yeast and Drosophila cells, e.g. Drosophila Schneider line 2 (S2) cells. Any eukaryotic cell known by one of skill in the art to be useful for expressing a recombinant polypeptide, without limitation, can be used to express the delivery constructs.

### 6.4 Antibodies

According to the invention, happyhour, or its fragments thereof, may be used as an immunogen to generate antibodies which immunospecifically bind happyhour polypeptides. Such antibodies include, but are not limited to, polyclonal, monoclonal, single chain monoclonal, recombinant, chimeric, humanized, mammalian, or human antibodies.

In some embodiments, antibodies to a non-human happyhour are produced. In certain embodiments, antibodies to rat happyhour are produced. In other embodiments, antibodies to human happyhour are produced. In another embodiment, antibodies are produced that specifically bind to a protein the amino acid sequence of which consists of SEQ ID NO:1 OR 3. In another embodiment, antibodies to a fragment of non-human happyhour are produced. In another embodiment, antibodies to a fragment of rat happyhour are produced. In another embodiment, antibodies to a fragment of human happyhour are produced. In a specific embodiment, fragments of happyhour, human or non-human, identified as containing hydrophilic regions are used as immunogens for antibody production. In a specific embodiment, a hydrophilicity analysis can be used to identify hydrophilic regions of happyhour, which are potential epitopes, and thus can be used as immunogens.

For the production of antibody, various host animals can be immunized by injection with native happyhour, or a synthetic version, or a fragment thereof. In certain embodiments, the host animal is a mammal. In some embodiments, the mammal is a rabbit, mouse, rat, goat, cow or horse.

For the production of polyclonal antibodies to happyhour, various procedures known in the art may be used. In some embodiments, rabbit polyclonal antibodies to an epitope of happyhour encoded by a sequence of SEQ ID NO:1 OR 3 or a subsequence thereof, can be obtained. Various adjuvants may be used to increase the immunological response, depending on the host species. Adjuvants that may be used according to the present invention include, but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, CpG-containing nucleic acids, and potentially useful human adjuvants such as BCG (bacille Calmette-Guerin) and Corynebacterium parvum.

For preparation of monoclonal antibodies directed toward a happyhour polypeptide, any technique that provides for the production of antibody molecules by continuous cell lines in culture may be used. For example, monoclonal antibodies may be prepared by the hybridoma technique originally developed by Kohler and Milstein, Nature 256:495-497 (1975), as well as the trioma technique, the human B-cell hybridoma technique (Kozbor et al., Immunol. Todaicy 4:72 (1983)), or the EBV-hybridoma technique (Cole et al., in Monoclonal Antibodies and Cancer Therapy, Alan R. Liss, Inc., pp. 77-96 (1985)).

Techniques for the production of single chain antibodies, as described in U.S. Pat. No. 4,946,778, can also be adapted to produce single chain antibodies specific to happyhour. An additional embodiment of the invention utilizes the techniques described for the construction of Fab expression libraries (Huse et al., Science 246:1275-1281 (1988)) to allow rapid and easy identification of monoclonal Fab fragments with the desired specificity for happyhour. Antibody fragments that contain the idiotype of the molecule can be generated by known techniques. For example, such fragments include but are not limited to: the F(ab′), fragment which can be produced by pepsin digestion of the antibody molecule; the Fab′ fragments which can be generated by reducing the disulfide bridges of the F(ab′), fragment, the Fab fragments which can be generated by treating the antibody molecule with papain and a reducing agent, and Fv fragments.

Techniques developed for the production of “chimeric” antibodies (Morrison et al., Proc. Natl. Acad. Sci. U.S.A. 81:6851-6855 (1984); Neuberger et al., Nature 312:604-608 (1984); Takeda et al., Nature 314:452-454 (1985)) can also be used. For example, nucleic acid sequences encoding a mouse antibody molecule specific to happyhour are spliced to nucleic acid sequences encoding a human antibody molecule.

In addition, techniques have been developed for the production of humanized antibodies, and such humanized antibodies to happyhour are within the scope of the present invention. See, e.g., Queen, U.S. Pat. No. 5,585,089 and Winter, U.S. Pat. No. 5,225,539. An immunoglobulin light or heavy chain variable region consists of a “framework” region interrupted by three hypervariable regions, referred to as complementarity determining regions (CDRs). The extent of the framework region and CDRs have been precisely defined. See, Sequences of Proteins of Immunological Interest, Kabat, E. et al., U.S. Department of Health and Human Services (1983). Briefly, humanized antibodies are antibody molecules from non-human species having one or more CDRs from the non-human species and a framework region from a human immunoglobulin molecule.

Human antibodies may be used and can be obtained by using human hybridomas (Cote et al., Proc. Natl. Acad. Sci. U.S.A., 80:2026-2030 (1983)) or by transforming human B cells with EBV virus in vitro (Cole et al., in Monoclonal Antibodies and Cancer Therapy, Alan R. Liss, pp. 77-96 (1985)).

In the production of antibodies, screening for the desired antibody can be accomplished by techniques known in the art, e.g. ELISA (enzyme-linked immunosorbent assay), RIA (radioimmunoassay) or RIBA (recombinant immunoblot assay). For example, to select antibodies which recognize a specific domain of happyhour, one may assay generated hybridomas for a product which binds to a happyhour fragment containing such domain. For selection of an antibody that specifically binds a first happyhour homologue but which does not specifically bind a second, different happyhour homologue, one can select on the basis of positive binding to the first happyhour homologue and a lack of binding to the second happyhour homologue.

Antibodies specific to a domain of happyhour or a homologue thereof are also provided. The foregoing antibodies can be used in methods known in the art relating to the localization and activity of the happyhour of the invention, e.g., for imaging these proteins, measuring levels thereof in appropriate physiological samples, in diagnostic methods, etc.

### 6.5 Methods of Screening for Modulators of Happyhour Activity

The present invention also provides methods of identifying an agent capable of modulating the activity of happyhour in a cell, tissue or organism of interest. An agent may modulate happyhour activity by affecting, for example: (1) the number of copies of the happyhour gene in the cell (amplifiers and deamplifiers); (2) increasing or decreasing transcription of the happyhour gene (transcription up-regulators and down-regulators); (3) by increasing or decreasing the translation of the happyhour mRNA into protein (translation up regulators and down regulators); or (4) by increasing or decreasing the activity of the happyhour protein (agonists and antagonists). To identify agents that are capable of modulating happyhour at the DNA, RNA, and protein levels, cells, tissues or organisms are contacted with a candidate compound and the corresponding change in happyhour DNA, RNA or protein may be assessed. For DNA amplifiers or deamplifiers, the amount of happyhour DNA may be measured. For those compounds that are transcription up-regulators and down-regulators, the amount of happyhour mRNA may be measured. Alternatively, the happyhour promoter sequence may be operably linked to a reporter gene, and potential transcriptional modulators of happyhour may be assayed by measuring reporter gene activity in the presence and absence of the compound. For translational up- and down-regulators, the amount of happyhour polypeptide may be measured. Alternatively, changes in happyhour biological activity, as measured by the techniques described below, may be an indirect indicator of the ability of a compound to modulate happyhour translation.

Happyhour activity of the methods described herein encompasses the biological activity of happyhour, which includes but is not limited to, modulation of ethanol sedation and modulation of the EGFR/ERK pathway. Methods for examining ethanol sedation and EGFR/ERK activity are known in the art, and include the exemplary methods described in the examples below.

In one embodiment, the cell or tissue or organism useful for the methods described herein expresses a happyhour polypeptide from an endogenous copy of the happyhour gene. In another embodiment, the cell or tissue or organism expresses a happyhour polypeptide following transient or stable transformation with a nucleic acid encoding a happyhour polypeptide of the present invention. Any cell known by one of skill in the art to be useful for expressing a recombinant polypeptide, without limitation, can be used to express a happyhour polypeptide useful for the methods described herein.

In one embodiment, the method of identifying an agent that is capable of modulating the activity of happyhour comprises determining a first level of happyhour activity in a cell or tissue that expresses a happyhour polypeptide, contacting said cell or tissue with a test agent, then determining a second level of happyhour activity in said cell or tissue. A difference in the first level and second level of happyhour activity is indicative of the ability of the test agent to modulate happyhour activity. In one embodiment, an agent may have agonistic activity if the second level of happyhour activity is greater than the first level of happyhour activity. In certain embodiments, agonistic activity comprises at least about a 2, 4, 6, 8, 10, or greater fold increase in the second level of happyhour activity compared to the first level of happyhour activity. In another embodiment, an agent may have antagonistic activity if the second level of happyhour activity is less than the first level of happyhour activity. In certain embodiments, antagonistic activity comprises at least about a 2, 4, 6, 8, 10, or greater fold decrease in the second level of happyhour activity compared to the first level of happyhour activity.

In another embodiment, the invention provides a method of identifying an agent that is capable of modulating the activity of happyhour in a cell or tissue or organism expressing a happyhour polypeptide, comprising contacting said cell or tissue organism with a test agent and determining a level of happyhour in said cell or tissue. The difference in this level and a standard or baseline level of happyhour activity in a comparable cell or tissue or organism, e.g. a control cell or tissue or organism not contacted with the test agent, is indicative of the ability of said test agent to modulate happyhour activity. In one embodiment, an agent may have agonistic activity if the level of happyhour activity in the cell or tissue or organism contacted with said agent is greater than the level of happyhour activity in the control cell or tissue or organism. In certain embodiments, agonistic activity comprises at least about a 2-, 4-, 6-, 8-, 10-, or greater fold increase in the level of happyhour activity of a cell or tissue or organism contacted with the test compound compared to the level of happyhour activity in the control cell or tissue or organism. In another embodiment, an agent may have antagonistic activity if the level of happyhour activity in the cell or tissue or organism contacted with said compound is less than the level of happyhour activity in the control cell or tissue or organism. In certain embodiments, antagonistic activity comprises at least about a 2-, 4-, 6-, 8-, 10-, or greater fold decrease in the level of happyhour activity of a cell or tissue or organism contacted with the test compound compared to the level of happyhour activity in the control cell or tissue or organism.

The present invention also provides methods of identifying agents that specifically bind to happyhour nucleic acids or polypeptides and thus have potential use as agonists or antagonists of happyhour. In certain embodiments, such agents may affect sensitivity to ethanol sedation or EGFR/ERK signaling. In a preferred embodiment, assays are performed to screen for agents having potential utility as modulators of drug-induced impairment, for example, ethanol sedation or locomotor impairment. The invention thus provides assays to detect compounds that specifically bind to happyhour nucleic acids or polypeptides. For example, recombinant cells expressing happyhour nucleic acids can be used to recombinantly produce happyhour polypeptides for use in these assays, e.g., to screen for compounds that bind to happyhour polypeptides. Said compounds (e.g., putative binding partners of happyhour) are contacted with a happyhour polypeptide or a fragment thereof under conditions conducive to binding, and compounds that specifically bind to happyhour are identified. Similar methods can be used to screen for compounds that bind to happyhour nucleic acids. Methods that can be used to carry out the foregoing are commonly known in the art.

Ethanol sedation can be assayed by any technique apparent to those of skill in the art. In certain embodiments, ethanol sedation can be assayed in Drosophila expressing a polypeptide of the invention. For instance, the Drosophila can be administered a test agent by standard techniques. For the assay, the Drosophila can be administered ethanol by standard techniques, for example, by exposure to ethanol vapors. Following administration, the Drosophila can be assayed visually for sedation. In certain embodiments, Drosophila that remain immobile, according to the judgment of a practitioner of skill in the art, are scored as sedated. Exemplary assays for ethanol sedation are provided in the examples below.

EGFR/ERK signaling can be assayed by any technique known to those of skill in the art. Exemplary assays for modulation of EGFR/ERK effects on Drosophila eye development are provided in the examples below.

In some embodiments, cell free assays utilizing a purified happyhour polypeptide may be performed to identify agents which modulate ethanol sedation or EGFR/ERK signalling. Putative modulators of happyhour biological activity may be identified by assaying happyhour kinase activity in the presence of varying concentrations of the agent and examining the extent of phosphate incorporation into a suitable substrate.

In various embodiments, the happyhour-modulating agent is a protein, for example, an antibody; a nucleic acid; or a small molecule. As used herein, the term “small molecule” includes, but is not limited to, organic or inorganic compounds (i.e., including heteroorganic and organometallic compounds) having a molecular weight less than 10,000 grams per mole, organic or inorganic compounds having a molecular weight less than 5,000 grams per mole, organic or inorganic compounds having a molecular weight less than 1,000 grams per mole, organic or inorganic compounds having a molecular weight less than 500 grams per mole, organic or inorganic compounds having a molecular weight less than 100 grams per mole, and salts, esters, and other pharmaceutically acceptable forms of such compounds. Salts, esters, and other pharmaceutically acceptable forms of such compounds are also encompassed.

By way of example, diversity libraries, such as random or combinatorial peptide or nonpeptide libraries can be screened for agents that specifically bind to happyhour. Many libraries are known in the art that can be used, e.g., chemically synthesized libraries, recombinant (e.g. phage display libraries), and in vitro translation-based libraries.

Examples of chemically synthesized libraries are described in Fodor et al., Science 251:767-773 (1991); Houghten et al., Nature 354:84-86 (1991); Lam et al., Nature 354:82-84 (1991); Medynski, Bio/Technology 12:709-710 (1994); Gallop et al., J. Medicinal Chemistry 37(9):1233-1251 (1994); Ohlmeyer et al., Proc. Natl. Acad. Sci. U.S.A. 90:10922-10926 (1993); Erb et al., Proc. Natl. Acad. Sci. U.S.A. 91:11422-11426 (1994); Houghten et al., Biotechniques 13:412 (1992); Jayawickreme et al., Proc. Natl. Acad. Sci. U.S.A. 91:1614-1618 (1994); Salmon et al., Proc. Natl. Acad. Sci. U.S.A. 90:11708-11712 (1993); PCT Publication No. WO 93/20242; and Brenner and Lerner, Proc. Nail. Acad. Sci. U.S.A. 89:5381-5383 (1992).

Examples of phage display libraries are described in Scott and Smith, Science 249:386-390 (1990); Devlin et al., Science, 249:404-406 (1990); Christian, R. B., et al., J. Mol. Biol. 227:711-718 (1992)); Lenstra, J. Immunol. Meth. 152:149-157 (1992); Kay et al., Gene 128:59-65 (1993); and PCT Publication No. WO 94/18318, published Aug. 18, 1994. In vitro translation-based libraries include but are not limited to those described in PCT Publication No. WO 91/05058, published Apr. 18, 1991; and Mattheakis et al., Proc. Natl. Acad. Sci. U.S.A. 91:9022-9026 (1994).

By way of examples of non-peptide libraries, a benzodiazepine library (see e.g., Bunin et al., Proc. Natl. Acad. Sci. U.S.A. 91:4708-4712 (1994)) can be adapted for use. Peptoid libraries (Simon et al., Proc. Natl. Acad. Sci. U.S.A. 89:9367-9371 (1992)) can also be used. Another example of a library that can be used, in which the amide functionalities in peptides have been permethylated to generate a chemically transformed combinatorial library, is described by Ostresh et al., Proc. Natl. Acad. Sci. U.S.A. 91:11138-11142 (1994).

Screening the libraries can be accomplished by any of a variety of commonly known methods. See, e.g., the following references, which disclose screening of peptide libraries: Parmley and Smith, Adv. Exp. Med. Biol. 251:215-218 (1989); Scott and Smith, Science 249:386-390 (1990); Fowlkes et al., Bio/Techniques 13:422-427 (1992); Oldenburg et al., Proc. Natl. Acad. Sci. U.S.A. 89:5393-5397 (1992); Yu et al., Cell 76:933-945 (1994); Staudt et al., Science 241:577-580 (1988); Bock et al., Nature 355:564-566 (1992); Tuerk et al., Proc. Natl. Acad. Sci. U.S.A. 89:6988-6992 (1992); Ellington et al., Nature 355:850-852 (1992); U.S. Pat. No. 5,096,815, U.S. Pat. No. 5,223,409, and U.S. Pat. No. 5,198,346; Rebar and Pabo, Science 263:671-673 (1993); and PCT Publication No. WO 94/18318, published Aug. 8, 1994.

In a specific embodiment, screening can be carried out by contacting the library members with happyhour polypeptide (or nucleic acid) immobilized on a solid phase and harvesting those library members that bind to the protein (or nucleic acid). Examples of such screening methods, termed “panning” techniques are described by way of example in Parmley and Smith, Gene 73:305-318 (1988); Fowlkes et al., Bio/Techniques 13:422-427 (1992); PCT Publication No. WO 94/18318; and in references cited herein above.

In another embodiment, the two-hybrid system for selecting interacting proteins in yeast (Fields and Song, Nature 340:245-246 (1989); Chien et al., Proc. Natl. Acad. Sci. U.S.A. 88:9578-9582 (1991)) can be used to identify molecules that specifically bind to happyhour protein or an analog thereof.

In another embodiment, screening can be carried out by creating a peptide library in a prokaryotic or eukaryotic cell, such that the library proteins are expressed on the cells' surface, followed by contacting the cell surface with happyhour and determining whether binding has taken place. Alternatively, the cells are transformed with a nucleic acid encoding happyhour, such that happyhour is expressed on the cells surface. The cells are then contacted with a potential agonist or antagonist, and binding, or lack thereof, is determined. In a specific embodiment of the foregoing, the potential agonist or antagonist is expressed in the same or a different cell such that the potential agonist or antagonist is expressed on the cells' surface.

As would clearly be understood by a person of ordinary skill in the art, any and/or all of the embodiments disclosed herein for identifying an agent, drug, or compound that can modulate the activity of happyhour, including such procedures that incorporate rational drug design, as disclosed herein, can be combined to form additional drug screens and assays, all of which are contemplated by the present invention.

### 7. EXAMPLES

The invention is illustrated by the following examples which are not intended to be limiting in any way.

### 7.1 Example 1

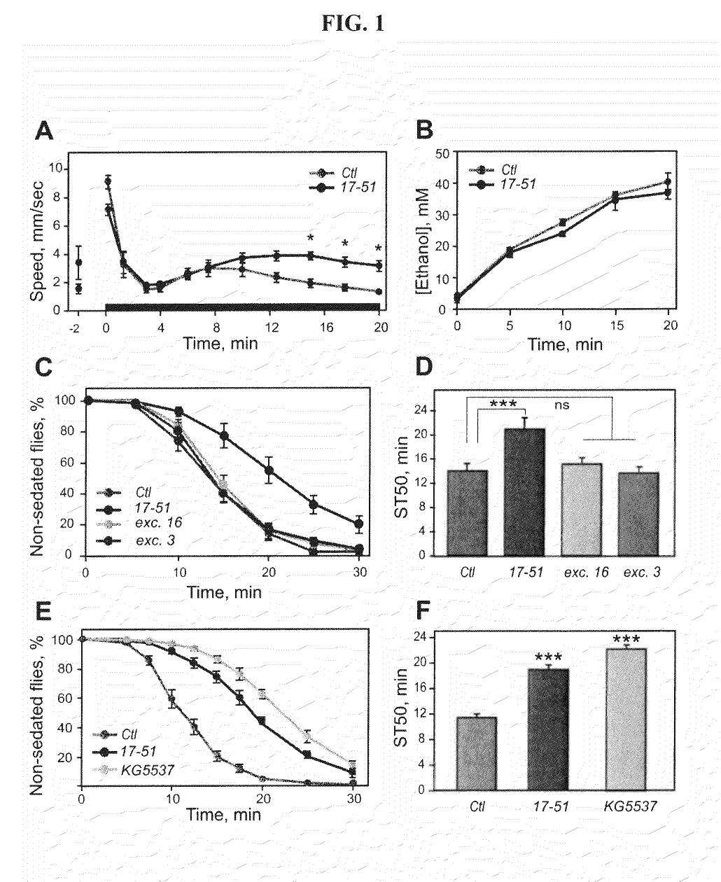

In an effort to identify novel molecules mediating the sedative responses to ethanol in Drosophila, we screened a collection of strains carrying the P{GawB} transposable element using a locomotor tracking device (Wolf et al., 2002). When exposed to a relatively high concentration of ethanol, Drosophila exhibit a fast and transient increase in their locomotor response (a startle response to the smell of ethanol), followed by a decrease in locomotor activity that is associated with the gradual loss of postural control and, finally, akinesis (sedation) (Wolf et al., 2002; FIG. 1A).

Genetic Screen and Selection of Controls

Approximately 850 P[GAL4] insertions (carrying the GawB element) in the w Berlin genetic background were screened as homozygotes in a “booz-o-mat” at a 100 U ethanol vapor/50 U air concentration as described below. After each day of screening, each fly line's ethanol-induced locomotor tracking profile was compared to the mean tracking profile of the day. Lines that were judged to have a locomotor tracking phenotype if they differed by at least 2 standard deviations from the mean at two or more consecutive time points. The 11 lines that retained their mutant phenotypes after two or more retests were selected for backcrossing for five generations to the parental w Berlin strain and retested after outcrossing. In addition, 32 control lines, including line 8-165 used in this paper, exhibiting tracking profiles similar to the mean tracking profile of the screened population over multiple days, were also backcrossed to the parental w Berlin strain in order to generate a set of control lines. Of the 11 outcrossed mutant lines, five, including 17-51 (hppy) retained their mutant phenotypes.

Fly Stocks

Flies were raised on standard cornmeal and molasses food at 25° C. and 70% relative humidity. All experiments were performed on 2-5 day old males at 20° C., utilizing 25 males for each behavioral run. All genotypes were tested across multiple days. All lines tested in behavioral experiments were in the same genetic background (w Berlin) with the exception of UAS-spi^{SEC} and UAS-dRaf^{GOF}, which were in the w1118 background isogenic for chromosomes II and III. Lines 17-51 (lippy) and control line 8-165 were obtained through the screen described above. Excisions of 17-51 were carried out through standard genetic crosses using the {delta 2-3} jump-starter chromosome (Robertson et al., 1988, Genetics 118:461-470). Elav-GAL4^{c155}, Tub-GAL4, GMR-GAL4, and hppy^{KG5537} flies were obtained from the Drosophila Stock Center (Bloomington, Ind.). S^{d01624} flies were obtained from the Exelixis Collection at the Harvard Medical School (Boston, Mass.). Elav-GAL4^{3El} flies were obtained from S. Sweeney (Davis et al., 1997, Neuron 19:561-573). Mutant Rho-l^{A0544} (Iks) was obtained from Tim Tully (Dubnau et al. 2003, Curr Biol 13:286-296). JNK pathway lines used include UAS-Bsk^{WT} (Boutros et al., 1998, Cell 94:109-118), UAS-Bsk^{DN} (Adachi-Yamada et al., 1999a, Nature 400:166-169), UAS-hep^{ACT} (Weber et al., 2000, Development 127:3619-3629), UAS-dJun^{DN} (Eresh et al., 1997, EMBO J 16:2014-2022), UAS-dJun-RNAi (Jindra et al., 2004, Embo J 23:3538-3547), and UAS-dJun^{WT} (Eresh et al., 1997). EGF pathway lines used include UAS-spi^{SEC} (Ghiglione et al., 2003, Development 130:4483-4493), UAS-Egfr^{WT} (Freeman, 1996, Cell 87:651-660), UAS-Egfr^{DN} (Freeman, 1996), UAS-dRaf^{GOF} (Brand and Perrimon, 1993, Development 118:401-415), UAS-rl^{ACT} (Ciapponi et al., 2001, Genes Dev 15:1540-1553), UAS-yan (Rebay and Rubin, 1995, Cell 81:857-866), and UAS-yan^{ACT} (Rebay and Rubin, 1995). P38 pathway lines used include D-p38a1 (Craig et al., 2004, EMBO Rep 5:1058-1063) and UAS-dp38b^{DN} (Adachi-Yamada et al., 1999b, Mol Cell Biol 19:2322-2329).

Lethality Enhancement Test

Five by five crosses in vials were set up and raised at 25° C. For these crosses, GMR-GAL4 or GMR-GAL4; UAS-hppy^{RB1} virgins were crossed to UAS-yan^{ACT}/CyO or UAS-egfr^{DN}/CyO males. Parents were cleared on day 2 and progeny were scored for the presence or absence of the CyO balancer chromosome on day 15. By Mendelian genetics, the number of progeny bearing the CyO balancer chromosome should make up 50% of the progeny in each cross. Percent lethality for each of these four genotypes: [1.) GMR-GAL4, UAS-yan^{ACT} 2.) GMR-GAL4, UAS-yan^{ACT}; UAS-hppy^{RB1} 3.) GMR-GAL4, UAS-egfr^{DN} and 4.) GMR-GAL4, UAS-egfr^{DN}, UAS-hppy^{RB1}] was calculated using the following equation: % lethality=(1-(# of non-Cy winged progeny/# of Cy winged progeny))×100%.

Behavioral Assays

Locomotor Tracking Assay

Locomotor tracking assays were performed in the “booz-o-mat” as described previously (Wolf et al., 2002). Briefly, twenty-five 2-4 day old males of each genotype were introduced to the chambers in the booz-o-mat. Flies were allowed to equilibrate in humidified air for 10 minutes before digital camera filming commenced. The motion of the flies was then recorded for 2 minutes in humidified air, followed by 21 minutes in an ethanol/air mixture of 50 U ethanol/100 U air. Films were then analyzed with a modified version of DIAS 3.2 (Solltech, Oakdale, Iowa) and the average speed of the flies was plotted as a function of time.

Ethanol Sedation Assay

Twenty-five 2-4 day old males of each genotype were introduced to the chambers in the booz-o-mat. After being given 12 minutes of humidified air to equilibrate to the apparatus, the flies were given a continuous stream of ethanol vapors (110 U EtOH/40 U air) for 30 minutes. During this thirty period time period, flies were visually assayed for sedation at 10 time points. At each time point, the flies were given a mechanical stimulus (each tube containing flies was twirled within each booz-o-mat chamber) and the number of flies that were lying immobile at the bottom of each tube post twirling were scored as being sedated. The assayer was blinded to the identity of the genotypes for the course of the sedation assay. The time to 50% sedation was determined by linear interpolation between the two points flanking the median for each set of 25 flies tested, as previously described (Rothenfluh et al., 2006).

Ethanol Absorption Assay

Twenty-five flies of each genotype were exposed in quadruplicate to an ethanol/humidified air mixture of 100/50 U for 0, 5, 10, 15, or 20 minutes in perforated test tubes in the booz-o-mat apparatus. Following exposure to ethanol, flies were frozen on dry ice and homogenized in 500 μl of 50 mM Tris-HCl (pH 7.5). Ethanol assays were then performed on the fly homogenates as previously described (Moore et al., 1998).

Immunohistochemistry

To visualize the adult CNS expression patterns for various GAL4 lines, e.g. dilp2-GAL4, TH-GAL4, Ddc-GAL4, and GAL4¹⁷⁻⁵¹, GAL4 virgins were crossed to UAS-GFP T2, UAS-Tau GFP males (double transgenic stock created by F. Wolf, UCSF) or UAS-GFP T2, UAS-Tau GFP; UAS-egfr^{WT} males. Brains and ventral nerve cords were dissected from 2-4 day old adult male progeny in 1×PBS, fixed in 4% paraformaldehyde for 20 minutes, then washed in 1×PBS. GFP labeling was achieved by incubating specimens in a 1:200 dilution of a rabbit anti-GFP antibody (Clontech, Mountain View, Calif.) and with a FITC-coupled goat-anti-rabbit antibody, diluted 1:500 (Molecular Probes, Eugene, Oreg.). Neuropil labeling was achieved by incubating specimens in a 1:10 dilution of Nc82 antibody (Laissue et al., 1999) and with a Cy3 coupled goat anti-mouse antibody, diluted 1:500 (Molecular Probes, Eugene, Oreg.). Specimens were mounted in Vectashield mounting medium (Vector Laboratories, Burlingame, Calif.) and analyzed with a Leica confocal microscope with Leica Confocal Software Version 2.5.

Molecular Biology

Characterization of hppy and transgene construction: The genomic DNA flanking the 17-51 (hppy) insertion was isolated using inverse PCR. Comparisons with the genome sequence of Drosophila on Flybase, which is funded by a grant from the National Human Genome Research Institute at the U.S. National Institutes of Health and supported by the British Medical Research Council and the Indiana Genomics Initiative, revealed that the insertion was located 10 base pairs upstream of the first exon of CG7097. This finding was confirmed by PCR analysis (data not shown). The UAS-lippy transgene, UAS-hppy^{RB}, was generated by cloning the EST RH10407, which encodes the full length CG7097-RB transcript, into the pUAST vector (Brand and Perrimon, 1993). This transgene was injected into w Berlin flies and two independent insertions were obtained, UAS-hppy^{RB1} and UAS-hppy^{RB2}, yielding different levels of hppy-RB expression (described in Results).

Star Complementation Assay

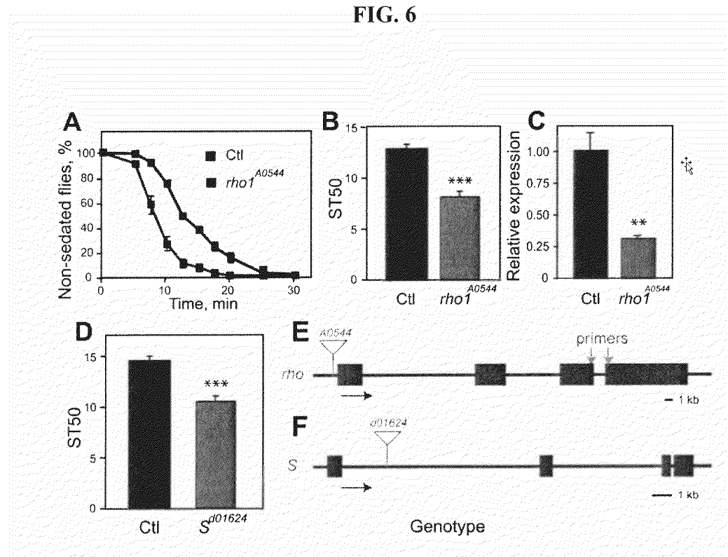

S^{d01624} homozygous virgins were crossed to a known null allele of Star, Sl/CyO, (Drosophila Stock Center, Bloomington, Ind.), progeny were scored for the presence or absence of the CyO balancer chromosome, and % lethality was calculated as described above. S^{d01624}/S^{l} trans-heterozygotes were rare to recover, with a % lethality of 91.6%, consistent with our hypothesis that S^{d01624} flies are Star hypomorphs. Exemplary results are presented in FIG. 6.

Scanning Electron Microscopy

Scanning electron microscopy was carried out as described previously (Kimmel et al., 1990). All images were taken at 130× magnification.

Statistics

Statistical significance was established using either Student's t-tests assuming equal variance or one-way analysis of variance (ANOVA) tests followed by post-hoc Newman-Keuls testing using GraphPad Prism software, Version 4 (Graphpad, San Diego, Calif.). Error bars in all experiments represent the standard error of the mean (SEM).

Results

In our screen, we isolated one mutant, line 17-51, that displayed resistance to ethanol-induced sedation as measured in the locomotor tracking system (FIG. 1A). Direct observations of sedation responses over a 30 minute ethanol exposure using a modified loss-of righting (LOR) assay (Rothenfluh et al., 2006), revealed that 17-51 mutants do indeed display a marked resistance to ethanol-induced sedation compared to controls (FIG. 1C, D) This is not simply due to a decrease in ethanol pharmacokinetics in 17-51 flies, as ethanol absorption over various lengths of ethanol exposure were normal in 17-51 flies (FIG. 1B). In addition, 17-51 flies show normal locomotor behavior and negative geotaxis.

### 7.2 Example 2

### Identification of Happyhour

The genomic DNA flanking the 17-51 (hppy) insertion was isolated using inverse PCR. Comparisons with the genome sequence of Drosophila on Flybase revealed that the insertion was located 10 base pairs upstream of the first exon of CG7097. This finding was confirmed by PCR analysis. The inverse PCR analysis revealed that the P{GawB} element in 17-51 is inserted 10 bp upstream of the first exon of the gene CG7097. We decided to name the CG7097 gene happyhour, since mutations in the gene result in flies being able to imbibe significantly more alcohol than controls before succumbing to its sedating effects. The transposon inserted in hppy^{KG5537} is responsible for the sedation resistance phenotype, as precise excisions of the element reverted the mutant phenotype (FIG. 1C, D). Database searches (flybase) revealed the presence of an additional P-element insertion near the happy¹⁷⁻⁵¹ insertion. When tested in the LOR assay, this mutant, hppy^{KG5537}, also showed resistance to ethanol-induced sedation (FIG. 1E, F). In addition, complementation assays between hppy¹⁷⁻⁵¹ and hppy^{KG5537} revealed that these mutants fail to complement each other's ethanol sedation phenotype (data not shown).

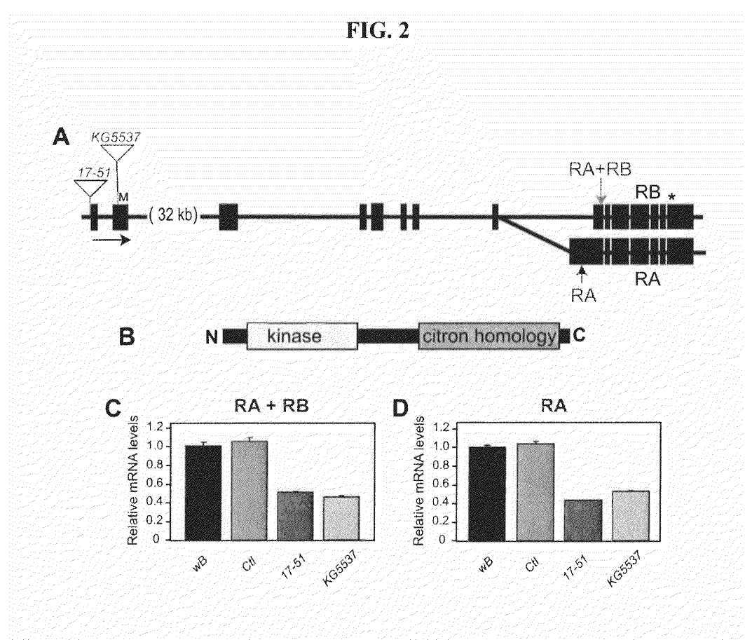

Genome analysis of the happyhour (CG7097) gene region (Flybase) indicated that the gene covers ˜48.5 kb, encoding two transcripts, hppy-RB and hppy-RA (FIG. 2A). Both transcripts, which are generated by alternative splicing of the eighth intron, share the same transcription start site, but the longer 5.1 kb hppy-RA transcript contains an additional 800 bp in its ninth exon not contained in the shorter 4.3 kb hppy-RB transcript. Our mutant strains contain P element insertions in the 5′ gene region of hppy. In hppy¹⁷⁻⁵¹, the transposon is inserted in the putative promoter region, 10 bp upstream of the first exon; the hppy^{KG5537} transposon is inserted in the first, non-coding exon (FIG. 2A). Both hppy-RA and hppy-RB transcripts are predicted to encode proteins containing an N-terminal serine/threonine kinase domain and a citron homology domain near the C-terminus (FIG. 2B; Simple Modular Architecture Research Tool; EMBL). The closest human homologs of HPPY are members of the germinal center kinase-1 (GCK-1) family of Ste20-related kinases, including GLK (germinal center-like kinase) and GCK itself (Dan et al., 2001, Trends Cell Biol 11:220-230; Findlay et al., 2007, Biochem. 1403:13-20). GCK-1 family members in other organisms have previously been shown to act as MAP4Ks in the JNK signaling pathway (Chen and Tan, 1999, Gene Ther Mol Biol 4:83-98), although in vitro studies of CG7097 (hppy) have failed to place this putative Drosophila MAP4K in the JNK signaling pathway (Findlay et al., 2007).

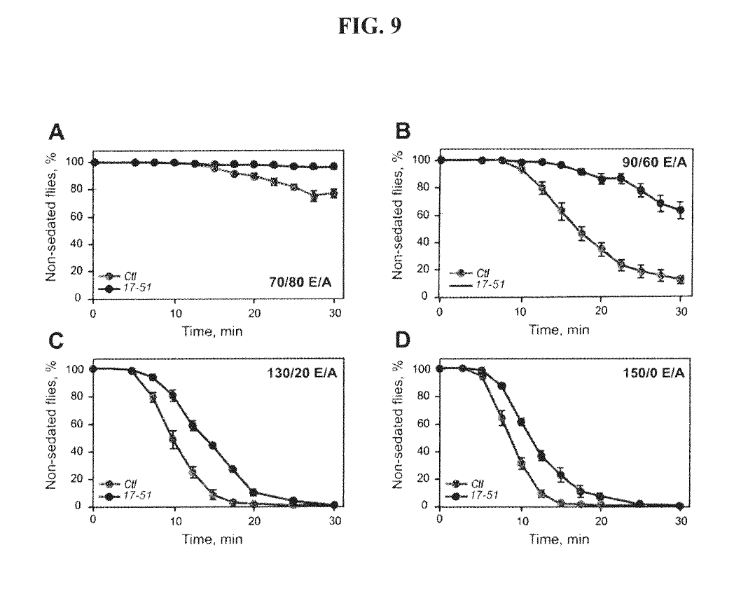

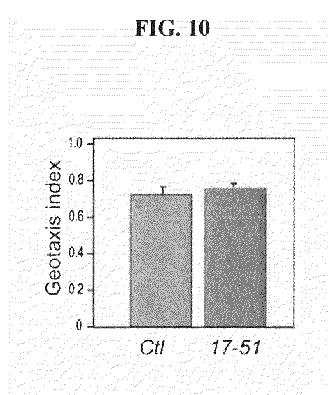

FIG. 9 shows that hppy¹⁷⁻⁵¹ flies are resistant to sedation when exposed to a broad range of ethanol concentrations. Direct observation of sedation responses over the 30-minute ethanol exposure using a modified loss-of righting (LOR) assay (Rothenfluh et al., 2006), confirmed that 17-51 mutants displayed a marked resistance to ethanol-induced sedation compared to controls (e.g., FIGS. 1C, D), a phenotype that was evident at all ethanol concentrations tested (e.g., FIG. 9). This was not simply due to altered ethanol pharmacokinetics, as ethanol absorption was normal in 17-51 flies (e.g., FIG. 1B). In addition, 17-51 flies show normal locomotor behavior and negative geotaxis, as shown in FIG. 10.

FIG. 10 reveals that hppy¹⁷⁻⁵¹ flies do not have defects in negative geotaxis. The negative geotaxis assay, which also measures locomotion and responsiveness to mechanical stimulation, was performed as described previously (Moore et al., 1998) with the following exceptions. The dimensions for the cylinder used were 22.5 cm in length by 2.7 cm in width. After loading the ten 2-4 day old flies of each genotype into a cylinder, the flies were banged down to the bottom and observed as they climbed to the top. At the end of each one minute period, the number of flies which had reached the top of the cylinder were counted and the flies were banged down once more. This process was repeated at 1 minute intervals for 5 minutes.

### 7.3 Example 3

### Real-Time Quantitative RT-PCR

To determine the effects the P-element insertions have on hppy expression, we assessed hppy transcript levels in our mutant and control strains by quantitative RT-PCR (QPCR).

Flies 2-4 days old were collected, frozen immediately in liquid nitrogen, then stored at −80° C. RNA was extracted from whole flies or isolated heads, as described for each experiment, by homogenization in Trizol Reagent (Invitrogen). Quantitative RT-PCR was performed as described in Tsai et al. (2004). The amplification primers and probe recognizing both CG7097-RA and CG7097-RB transcripts were: CG7097-RA&RB-For, CAGCGTTTTGGCATTCCATAA; CG7097-RA&RB-Rev, CGTCACCTCGCCATTGC; and CG7097-RA&RB-Probe, ATGCAGGGAAAGTC. The amplification primers and probe recognizing specifically the CG7097-RA transcript were: CG7097-RA-For, GTTGGCCACATGGGTATGG; CG7097-RA-Rev, GGTGCGC AGGTTGGACAT, and CG7097-RA-Probe, ATTTGGCATGGGTCTC. The amplification primers and probes for rhomboid-l expression analysis were obtained from Applied Biosystems. The assay IDs for the primer/probe sets used are: Dm01821933_g1 (rhomboid) and Dm02151827_g1 (Rp12), as well as Dm01821932_ml (rhomboid-1, more 5′) and Dm02151827_g1 (Rp132).

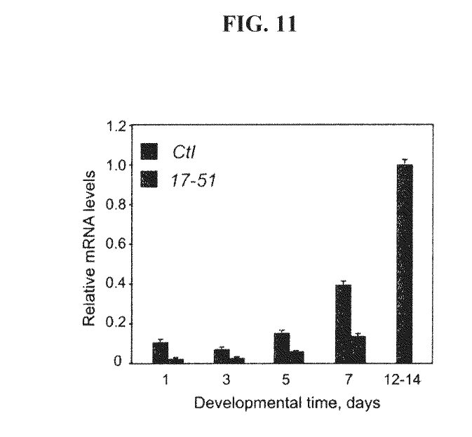

Using the primer and probe set recognizing both hppy-RB and hppy-RA transcripts, we found that the transcripts, we examined relative levels of hppy expression during various developmental stages as well as in adult flies. In control flies, hppy expression appeared relatively low during early development (embryos and 2^{nd} instar larvae), increased during later developmental stages (3^{rd} instar wandering larvae and pupae) and peaked in adults (e.g., FIG. 11). The relative expression of hppy in the hppy¹⁷⁻⁵¹ and hppy^{KG5537} mutants was reduced to approximately half that of controls in adult flies (e.g., FIG. 2C); hppy levels were decreased in hppy mutant flies compared to controls during development as well (e.g., FIG. 11). A similar reduction in hppy expression was seen in the mutants when using a primer and probe set recognizing specifically the hppy)-RA transcript (e.g. FIG. 2D). We were unable to generate a primer/probe set directed specifically against hppy-RB, since the entire sequence of hppy-RB is contained within the larger hppy-RA transcript. In summary, we have identified two mutations in the hppy locus that share an increased resistance to ethanol-induced sedation and show reduced levels of hppy transcripts.

FIG. 11 depicts hppy expression levels during development and adulthood. For assaying relative happyhour transcript levels during various life stages during development versus adult. RNA was extracted from staged embryos (approximately 16 hours post egg-laying), 2^{nd} instar larvae (3 days post egg-laying), wandering 3^{rd} instar larvae (5 days post egg-laying), and pupae (7 days post egg-laying). QPCR was performed on these samples as well as on 2-4 day old adult fly RNA as described in Tsai et al. (2004) using amplification primers recognizing both CG7097-RA and CG7097-RB transcripts. The amplification primers and probes for egfr expression analysis were obtained from Applied Biosystems. The assay IDs for the primer/probe sets used for egfr expression analysis are: Dm01841623_g1 (egfr) and Dm02151827_g1 (Rp132).

### 7.4 Example 4

### Behavioral Rescue of The Ethanol Resistance of Hppy Mutants

In order to conclusively demonstrate that the increased sedation resistance observed in hppy mutants was due to a decrease in hppy, expression, we attempted to rescue the mutant behavioral phenotype by expressing a UAS-hppy transgene in the hppy mutant background.

We generated a UAS-hppy^{RB} construct by inserting the complete hppy-RB cDNA sequence into the pUAST vector and introduced this transgene into hppy¹⁷⁻⁵¹ homozygous mutant flies. The UAS-hppy transgene was generated by cloning the EST RH10407, which encodes the full length CG7097-RB transcript, into the pUAST vector (Brand and Perrimon, 1993). The transgene was injected into w Berlin flies.

The hppy¹⁷⁻⁵¹ P[GAL4] insertion drives widespread GAL4 expression in tissues including the central nervous system, as visualized with a UAS-green fluorescent protein (UAS-GFP) reporter transgene.

hppy¹⁷⁻⁵¹ homozygous mutant flies carrying the UAS-hppy^{RB1} transgene have increased expression of specifically the hppy-RB transcript, as assayed by quantitative RT-PCR.

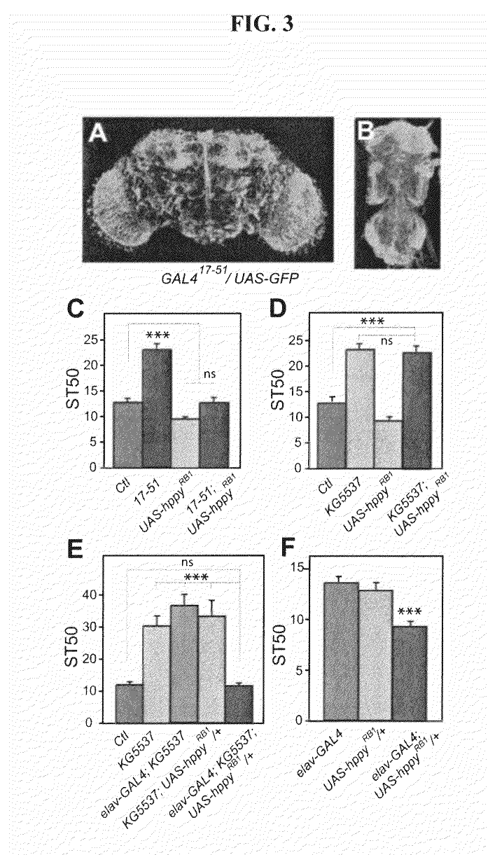

When tested in the LOR assay, these flies (hppy¹⁷⁻⁵¹/hppy¹⁷⁻⁵¹; UAS-hppy^{RB1}/UAS-hppy^{RB1}) displayed wild-type behavior (e.g., FIG. 3C), indicating complete rescue of the mutant phenotype by hppy-RB expression. Partial rescue was also achieved by using a second, more weakly expressed insertion of UAS-hppy^{RB} (UAS-hppy^{RB2}; e.g., FIG. 10). Importantly, introducing the UAS-hppy^{RB} trangenes into the hppy^{KG5537} homozygous mutant background, in which GAL4 is not expressed (see Methods), did not rescue the hppy^{KG5537} sedation resistance phenotype (e.g., FIG. 3D and data not shown). Finally, expression of an innocuous transgene, encoding inactive tetanus toxin light chain in the hppy¹⁷⁻⁵¹ homozygous mutant flies, failed to rescue the mutant phenotype (data not shown). These data confirm that the reduction in hppy expression is responsible for the resistance to ethanol-induced sedation observed in hppy mutant flies.

To determine whether expression of hppy specifically in the nervous system was sufficient to restore normal ethanol-induced sedation to hppy mutant flies, we expressed the UAS-hppy^{RB1} transgene in neurons using the elav-GAL4^{c155} driver in hppy^{KG5537} homozygous mutant flies. Indeed, neuronal expression of lippy-RB completely rescued the sedation resistance of hppy^{KG5537} flies (e.g., FIG. 3E). We next asked if neuronal overexpression of hppy would lead to enhanced sensitivity to the sedative effects of ethanol, the opposite effect caused by reduced hppy expression. Flies expressing the UAS-hppy^{RB1} transgene under the control of the pan-neuronal elav-GAL4^{c155} driver in an otherwise wild-type background showed a significant increase in sensitivity in the LOR assay (e.g., FIG. 3F). These data demonstrate that hppy functions in neurons to control ethanol-induced sedation, and that the pathway whose function is regulated by hppy can enhance or suppress the flies' response to the sedating effects of ethanol.

Discussion

We thus identified, through an unbiased genetic screen for Drosophila mutants with altered behavioral sensitivity to ethanol, a mutant in the gene happyhour (hppy). Loss of function of hppy results in increased resistance to ethanol-induced sedation, whereas neuronal overexpression of hppy causes the opposite effect, decreased resistance to sedation.

### 7.5 Example 5

### Perturbations of The JNK and P38 Signaling Pathways do not Affect Ethanol Induced Sedation

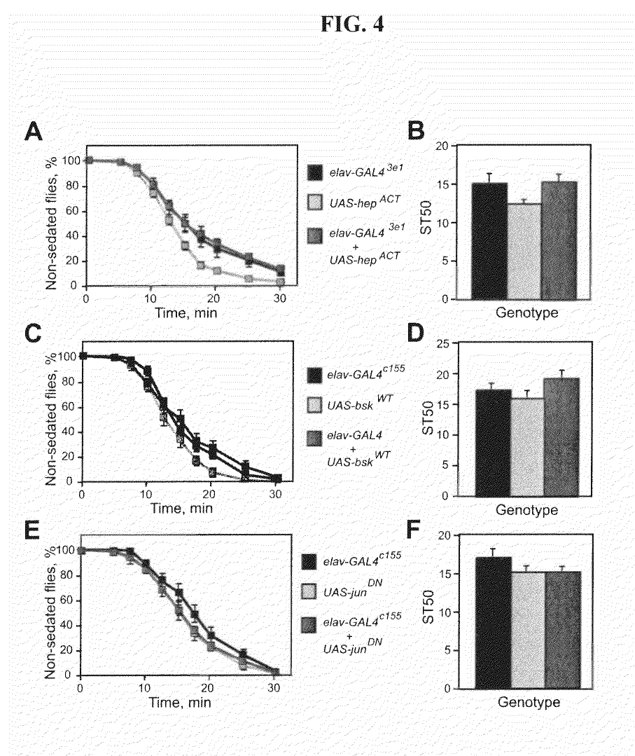

Since previous work had shown that a human homolog of hppy, germinal center kinase (GCK), activates the JNK pathway through MAP4K signaling (Pombo et al., 1995, Nature 377:750-754; Dan et al., 2001), we investigated whether perturbation of JNK pathway signaling in Drosophila would alter sensitivity to ethanol-induced sedation as measured in the LOR assay. To do this, we expressed various transgenes known to activate or inhibit the JNK pathway using the pan neuronal drivers elav-GAL4^{c155} and elav-GAL4^{3El} (elav-GAL4^{c155} expresses higher levels of GAL4 than elav-GAL4^{3El}). We found that neither activation nor inhibition of the INK signaling pathway altered the sensitivity of flies to ethanol-induced sedation, as measured in the ethanol LOR assay. For example, flies neuronally expressing a constitutively activated form of the JNKK hemipterous (hep) (using a UAS-hep^{ACT} transgene) showed wild-type sensitivity (FIG. 4A, B). Similarly, neither neuronal overexpression of the Drosophila homologue of JNK, basket (bsk, using a UAS-bsk^{WT} transgene), nor a dominant-negative form of JNK (using a UAS-bsk^{DN} transgene), altered LOR sensitivity (FIG. 4C, D and data not shown). Finally, neuronal overexpression or inhibition of the JNK pathway transcription factor dJUN, through expression of wild-type or a dominant-negative form of dJUN (using UAS-jun^{WT} or UAS-jun^{DN}, respectively) also failed to affect ethanol-induced sedation behaviors (FIG. 4E, F).

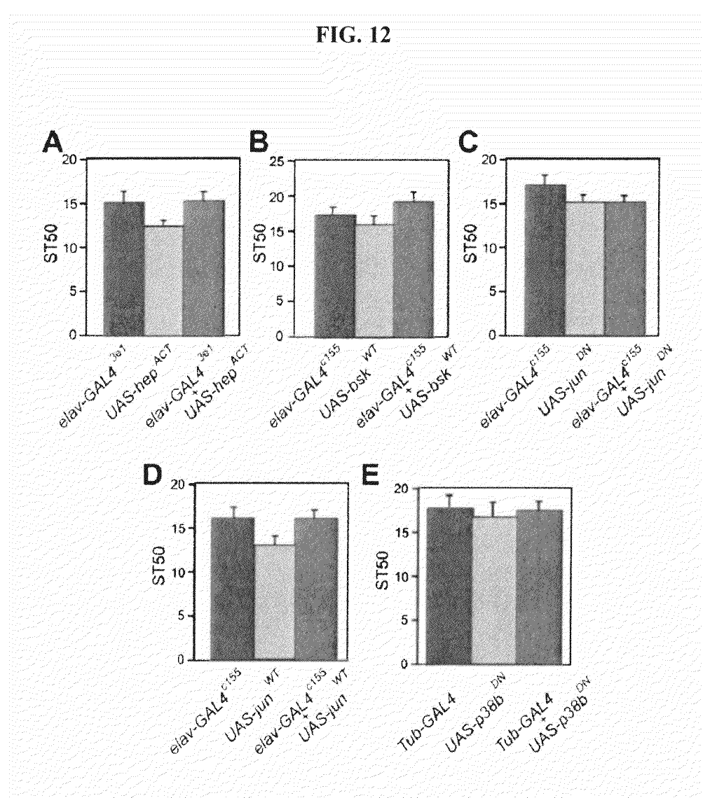

We also tested the effects of perturbing a second major MAPK pathway, the p38 pathway; these manipulations similarly had no significant effect on ethanol-induced sedation (e.g., FIG. 12).

Discussion

Hppy encodes a presumed MAP4 kinase with homology to GCK. To identify a signaling cascade that hppy may regulate, we tested whether neuronal perturbation of the major MAP kinase cascades would affect ethanol sensitivity in Drosophila. We found that manipulations that activate or inhibit the JNK or p3⁸ pathways did not affect ethanol-induced sedation.

### 7.6 Example 6

### Perturbations of Neuronal EGFR Signaling Alter Ethanol Sensitivity

Since manipulations of the JNK and p38 pathways failed to alter ethanol sensitivity, we tested the remaining MAPK signaling pathway, the ERK pathway, for its potential role in regulating ethanol sensitivity. We asked if perturbations of the ERK pathway, specifically the EGFR pathway, had an effect on ethanol-induced sedation by driving expression of various EGF pathway transgenes using the pan-neuronal drivers elav-GAL4^{c155} or elav-GAL4^{3El}. We also attempted to drive transgene expression with the ubiquitous driver Tub-GAL4, but found that this resulted in lethality in all cases except when driving the expression of a secreted form of the EGFR ligand encoded by the spitz (spi) gene, a condition that produced viable and healthy flies.

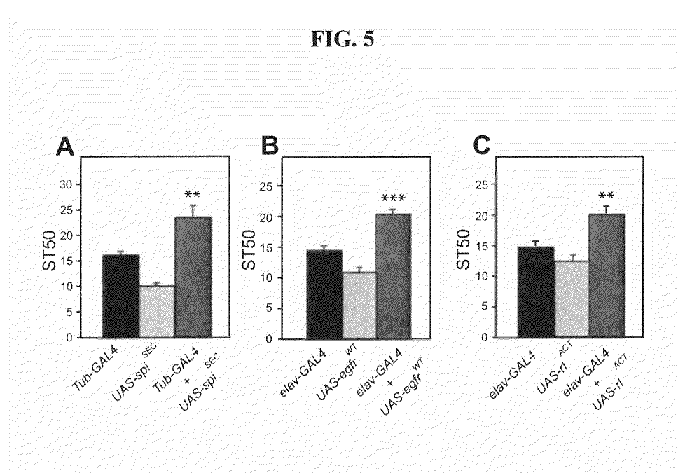

Interestingly, we found that manipulations that enhanced EGF signaling at several levels in the pathway potently increased resistance to ethanol-induced sedation, as measured in the LOR assay. Expression of secreted Spitz, by driving expression of a UAS-spi^{SEC} transgene using either Tub-GAL4 or elav-GAL4^{c155}, strongly increased resistance to ethanol-induced sedation (FIG. 5A, and data not shown). Marked resistance was also produced by driving neuronal expression of a wild-type EGFR transgene (UAS-egfr^{WT}, FIG. 5B), a gain-of-function Raf MAP3K (UAS-dRaf^{GOF}, data not shown), or a constitutively active form of the ERK rolled (rl) (UAS-rl^{ACT}, FIG. 5C). Conversely, a P element-induced loss-of-function mutation in rhomboid-l (rho-l), which codes for an enzyme that activates EGFR signaling through proteolysis of the ligand Spitz (Lee et al., 2001, Cell 107:161-171), induces the opposite effect, enhanced sensitivity to ethanol-induced sedation (FIG. 6B, C). In this allele, which carries a P element insertion in the promoter region of rho-l (FIG. 6A), mRNA levels are reduced to ˜30% of wild-type as measured by QPCR (FIG. 6D).

We also tested a mutant in Star (S), which encodes a chaperone required for intracellular trafficking of Spitz (Lee et al., 2001). S^{d01624} flies, which carry a P element insertion within the Star gene (e.g., FIG. 12B) that reduces Star function as ascertained by complementation analysis with a known null allele of Star, also showed enhanced sensitivity to ethanol-induced sedation (FIG. 6D).

Taken together, our data strongly supports a role for the EGFR pathway in regulating ethanol-induced sedation in Drosophila, where inhibition of the pathway leads to enhanced sensitivity to the sedating effects of ethanol, while activation of the pathway leads to the opposite phenotype, resistance to sedation.

Discussion

To identify a signaling cascade that hppy may regulate, we tested whether neuronal perturbation of the major MAP kinase cascades would affect ethanol sensitivity in Drosophila. In an example above, we found that manipulations that activate or inhibit the JNK or p38 pathways did not affect ethanol-induced sedation. In contrast, perturbations of the ERK pathway activated by the EGFR strongly altered the sensitivity of flies to the sedating effects of ethanol. Specifically, activation of the EGFR pathway in the nervous system resulted in strong resistance to ethanol, whereas inhibition of the pathway induced enhanced sensitivity.

### 7.7 Example 7

### Genetic Interactions Between Hppy And The EGFR Pathway

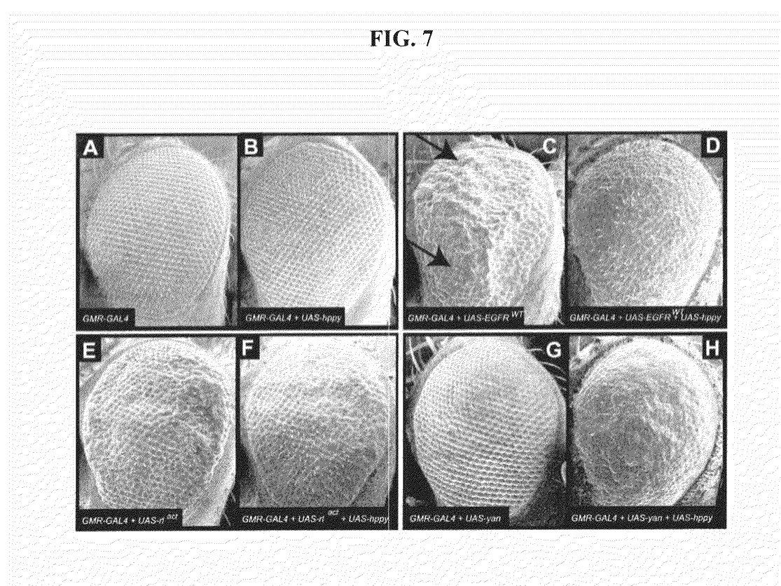

Based on our observations that enhanced EGFR signaling and loss of hppy function both lead to resistance to the sedative effects of ethanol, while reduced EGFR signaling and hppy overexpression lead to the opposite effect, enhanced sensitivity, we reasoned that hppy may function as an inhibitor of the EGFR pathway. In order to test this hypothesis, we resorted to the fly eye, where the developmental role of EGFR signaling has been thoroughly studied (reviewed in Dominguez et al. 1998, Current Biology 8:1039-1048). Specifically, we tested whether overexpression of hppy-RB could enhance or suppress the rough eye phenotypes induced by expression of EGFR pathway components using the GMR-GAL4 driver, which drives expression in developing retinal cells (Moses and Rubin, 1991, Genes and Development 5:583-593). Expression of UAS-hppy^{RB1} under the control of GMR-GAL4 resulted in an essentially wild-type eye phenotype (compare FIGS. 7A and 7B). Overexpressing the EGFR using UAS-Egfr^{WT} resulted in a very strong rough eye phenotype with prominent blistering in the dorsal anterior section of the eye (FIG. 7C). A rough eye phenotype was also observed when expressing a constitutively active form of the MAPK rolled, using the UAS-rl^{ACT} transgene, under the control of GMR-GAL4 (FIG. 7E). We found that hppy overexpression was able to suppress the rough eye and blistering phenotypes induced by overexpression of the EGFR (FIG. 7D). In contrast, expression of hppy did not affect the rough eye phenotype caused by expression of activated rolled (FIG. 7E, F). Having shown that hppy expression could suppress the rough eye phenotype induced by EGFR pathway activation, we asked whether hppy expression could enhance the rough eye phenotype caused by the expression of the transcription factor YAN, which acts downstream of rolled to inhibit the transcription of EGFR pathway activated genes (O'Neill et al., 1994, Cell 78:137-147). Indeed, while expression of wild-type yan, using the UAS-yan^{WT} transgene, produced an overall normal looking eye with an orderly arrangement of ommatidia (FIG. 7G), the combined expression of hppy and yan under GMR-GAL4 control produced a severe rough and “glossy” eye phenotype in which large swaths of ommatidia were absent (FIG. 7H), showing that hppy and yan act synergistically to inhibit downstream targets of the EGFR pathway. Taken together, our data implicates a role for hppy as an inhibitor of EGFR signaling, acting downstream of the EGFR, but upstream of the MAPK rolled.

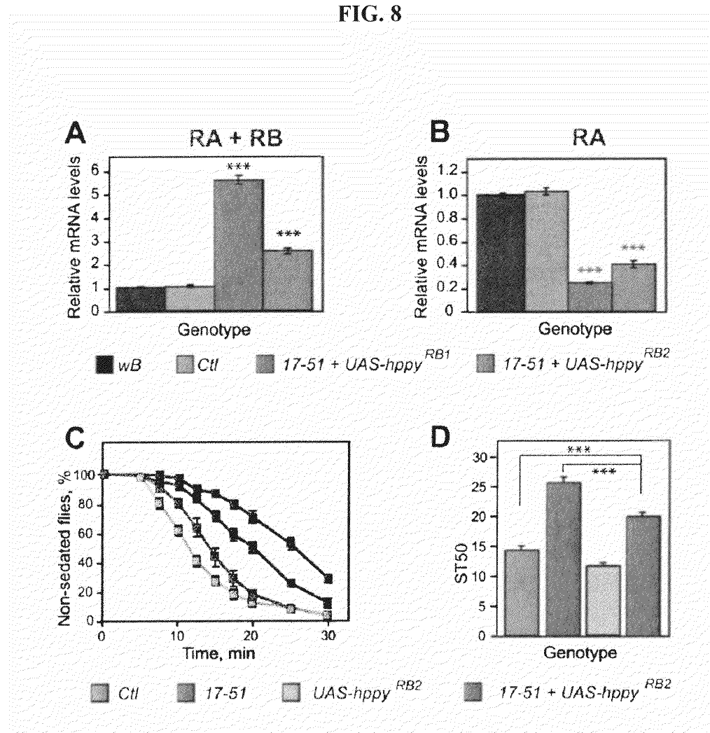

Interestingly, when expressing various EGFR pathway components with the GMR-GAL4 driver, we observed that expression of either the dominant negative form of the EGFR (UAS-Egfr^{DN}) or an activated form of yan (UAS-yan^{ACT}), resulted in reduced viability (FIG. 8). Interestingly, co-expression of hppy (UAS-hppy^{RB1}) potently enhanced the lethality of both GMR-GAL4 driven UAS-Egfr^{DN} and UAS-yan^{ACT}. Taken together, these data provide evidence that hppy modulates the EGFR pathway in its requirement for normal eye development and viability, and are consistent with hppy functioning as an inhibitor to the pathway, acting downstream of the EGFR and upstream of the ERK Rolled.

Thus, hppy can modulate EGFR signaling in a manner that is consistent with it acting as an inhibitor of the pathway, operating downstream of the EGFR but upstream of the ERK rolled.

Discussion

Happyhour Regulates Ethanol-Induced Sedation and EGFR Signaling in Drosophila

Utilizing a forward genetic approach to search for Drosophila mutants displaying altered responses to ethanol, we identified and characterized two P-element mutants in the CG7097/happyhour (hppy) gene region. We found that decreased hppy expression resulted in decreased sensitivity to the sedative effects of ethanol, as measured in a modified LOR assay, whereas neuronal overexpression of hppy caused the opposite effect. Through in situ hybridization and quantitative RT-PCR (data not shown), we found evidence for hppy expression in adult brains, and behavioral rescue experiments demonstrated that neuronal expression of hppy was sufficient to rescue the hppy sedation resistance phenotype. In addition, we found that complete rescue could be achieved by restoring expression of specifically the hppy-RB transcript, suggesting functional redundancy between the hppy-RB and hppy-RA transcripts or that the hppy-RA transcript may not be required for normal ethanol-induced sedation behaviors.

Like its mammalian homologs, the GCK-1 subfamily of Ste20 family kinases, both hppy transcripts are predicted to encode proteins that bear N-terminal serine/threonine kinase domains and C-terminal regulatory domains known as citron homology domains. In vitro studies of these homologs of hppy, including germinal center kinase (GCK) (Pombo et al., 1995), GCK-like kinase (GLK; Diener et al., 1997, Proc Natl Acad Sci USA 94:9687-9692), kinase homologous to SPS1/STE20 (KHS; Tung and Blenis, 1997, Oncogene 14:653-659), and hematopoietic progenitor kinase (HPK1; Kiefer et al., 1996, Embo J 15:7013-7025), have revealed that these GCK-1 subfamily members specifically activate the JNK signaling cascade, but not the ERK or p38 MAPK pathways. HPK1 (Hu et al., 1996, Genes Dev 10:2251-2264) and GLK (Diener et al., 1997) have both been shown to phosphorylate MAP3Ks in the JNK signaling pathway, positioning these GCK-1 subfamily kinases as MAP4Ks, situated upstream of the traditional three-tiered MAPK signaling cascade, but downstream of membrane signaling elements. While more distantly related Ste20 group kinases, such as those belonging to the GCK-VIII subfamily of thousand and one (TAO) kinases, have been shown to act as MAP3 kinases, activating both the JNK and the p38 stress-activated MAP kinase cascades (Hutchison et al., 1998, J Biol Chem 273:28625-28632; Chen and Tan, 1999; Yustein et al., 2000, Oncogene 19:710-718), until this study there have not been any reports of GCK kinases having a modulatory role on ERK signaling.

In this study we present evidence to show that HPPY, a presumed MAP4K in the GCK-1 subfamily of Step 20 kinases, can indeed modulate ERK signaling in a manner that is consistent with it acting as an inhibitor of ERK signaling functioning upstream of ERK itself but downstream of the EGFR. We find that HPPY can enhance and suppress the rough eye phenotypes brought about by EGFR/ERK perturbations as well as enhance the semi-lethality induced by expression of EGFR/ERK pathway inhibitors. In addition, decreasing levels of hppy completely suppressed the enhanced ethanol sensitivity brought about by neuronal EGFR downregulation. These findings are consistent with our hypothesis that hppy is epistatic to egfr, and that HPPY acts through the EGFR/ERK pathway to modulate ethanol-induced sedation in Drosophila.

An in vitro study of another GCK-1 subfamily kinase, HPK1, offers up an intriguing possibility (Anafi et al., 1997, J Biol Chem 272:27804-27811) of biochemical mechanism through which hppy inhibits EGFR/ERK signaling. In this study, the authors found that HPK1 physically associates with the EGFR adaptor protein Grb2 both in a yeast two-hybrid assay as well as in transfected mammalian cells. EGF stimulation recruits the Grb2/HPK1 complex to the autophosphorylated EGFR. This recruitment then leads to the tyrosine phosphorylation of HPK1, although the functional consequences of this phosphorylation are yet unknown. It would be interesting to determine whether such an association might exist between HPPY and members of the EGFR/ERK signaling cascade, and what consequences this association may have on the signaling of this MAP kinase pathway.

From our experiments we cannot rule out a role for hppy in regulating JNK signaling, although the lack of effect of JNK signaling perturbation on behavioral sensitivity to ethanol strongly suggests that hppy does not mediate its effects on ethanol-induced sedation through the JNK pathway. We also found that hppy mutant flies do not respond differently from controls when exposed to a variety of stress stimuli known to activate the JNK and p38 pathways, including oxidative stress, heat stress, and starvation (data not shown), further supporting the hypothesis that hppy is not involved in transducing signals through these stress-activated MAPK pathways. Indeed, in vitro studies in Hela cells support this hypothesis, demonstrating a lack of involvement of hppy in mediating JNK activation in response to stress stimuli such as osmotic stress and the protein synthesis inhibitor anisomycin (Findlay et al., 2007). Instead, Findlay et al. offer evidence that hppy can act as a nutrient sensor of amino acids and can stimulate phosphorylation of S6 kinase (S6K) through the mammalian target of rapamycin (mTOR) signaling pathway. The authors show that overexpression of wild-type hppy, but not a kinase-inactive mutant, can induce this S6K phosphorylation, demonstrating that hppy can in fact function as a kinase (Findlay et al., 2007). Interestingly, the mTOR signaling pathway, a key regulator of cell growth, is itself intimately regulated by inputs from the ERK signaling pathway (Sarbassov et al., 2005, Curr Opin Cell Biol 17:596-603), suggesting the attractive possibility that hppy may exert its stimulatory role on the mTOR pathway via its effects on EGFR/ERK pathway signaling.

The EGFR/ERK pathway mediates ethanol-induced sedation in Drosophila

The ERK signaling cascade has traditionally been studied for its roles in regulating a variety of developmental processes, including cell division, survival, and differentiation (reviewed in Chen et al., 2001; Pearson et al., 2001). More recent research, however, has revealed that the ERK pathway also plays important roles in mediating synaptic plasticity in post-mitotic neurons (Sweatt, 2004) (Mazzucchelli and Brambilla, 2000). For instance, establishment of long-term potentiation requires the activation of ERK, and inhibition of ERK signaling has been shown to disrupt both hippocampal- and amygdala-dependent learning (Brambilla et al., 1997, Nature 390:281-286.; Atkins et al., 1998, Nat Neurosci 1:602-609; Selcher et al., 1999, Learn Mem 6:478-490). In addition, the EGFR/ERK pathway has been implicated in the regulation of circadian rhythms via its activation by transforming growth factor-α in the suprachiasmatic nucleus (Kramer et al., 2001; Hao and Schwaber, 2006, Brain Research 1088:45-48). Recently, an interesting literature has documented the inhibitory effects of ethanol on the EGFR/ERK pathway in neurons. Ethanol administration inhibits EGFR and ERK phosphorylation both in neuronal cell cultures (Kalluri and Ticku, 2003, Neurochem Res 28:765-769; Chandler and Sutton, 2005; Ma et al., 2005) as well as in mouse and rat brains (Kalluri and Ticku, 2002, Eur J Pharmacol 451:51-54; Sanna et al., 2002, Brain Res 948:186-191).

The present invention provides a previously undocumented role for the EGFR/ERK pathway in mediating the behavioral responses to ethanol in Drosophila. Neuronal manipulations that activate the EGFR/ERK pathway result in decreased sensitivity to the sedative effects of ethanol, whereas inhibition of the pathway results in increased sensitivity to ethanol-induced sedation. These effects were seen through manipulations of various components of the EGFR/ERK pathway, including ERK itself, the MAP3K (dRaf), the EGFR, the EGF receptor ligand Spitz, and the enzyme, Rhomboid-1, that processes Spitz into its active form. In contrast, we find no evidence for the other two major MAPK pathways, the JNK and p38 pathways, in mediating the sedative response to ethanol, suggesting a specific role for the ERK pathway. The EGFR/ERK pathway joins other growth factor pathways, such as the insulin, glial cell line-derived neurotrophic factor (GDNF), and brain-derived neurotrophic factor (BDNF) pathways, as regulators of the behavioral response to ethanol (Janak et al., 2006, Alcohol Clin Fxp Res 30:214-221).

The pathways through which the EGFR/ERK cascade detects ethanol signals and how it might transduce those signals into a behavioral response remain unknown. The ERK cascade is activated through a variety of sources, including growth factors, serum, and ligands for heterotrimeric G protein-coupled receptors (reviewed in Chen et al., 2001). Interestingly, recent studies have implicated a role for both GABAA and NMDA receptors in ethanol-mediated inhibition of ERK in vitro, suggesting that ethanol may influence ERK activation state via signaling through these receptors (Kalluri and Ticku, 2002, 2003). The targets of ERK signaling are multitudinous, and our knowledge of the substrates of ERK signaling is ever expanding. In addition to phosphorylating and activating various transcription factors, ERK also targets various protein kinases, voltage-gated ion channels, and various second messenger systems such as cytosolic phospholipase A2 (Atkins et al., 1998; Chen et al., 2001). One alluring target of ERK signaling is the phosphodiesterase (PDE) 4D3, a cyclic adenosine monophosphate (cAMP)-specific phosphodiesterase which negatively regulates cAMP/PKA signaling by degrading intracellular cAMP (Chen et al., 2001). The cAMP signaling pathway, in turn, has been shown to regulate the ERK pathway, with inhibition of ERK signaling being induced by C-RAF phosphorylation by PKA (Dumaz and Marais, 2005, Febs J 272:3491-3504). Various studies in both Drosophila and mammals have demonstrated a role for the cAMP/PKA signaling cascade in mediating the behavioral responses to ethanol (Moore et al. 1998; Park et al., 2000; Thiele et al., 2000; Mass et al., 2005), raising the exciting possibility that “crosstalk” between these two conserved pathways may be integral to regulating ethanol induced sedation behaviors.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### SEQUENCE LISTING

SEQ ID NO: 1
MAAAHSHHNANMLSSDISRRNPQDEYELIQKIGSGTYGDVYKAKRIQSNE
LAAIKVIKLEPSDDIQIIQQEIIMMRDCRHPNIIAYYGSYLRRDKLWICM
EFCGGGSLQDIYQVTGPLTEVQIAYMCRETLKGLEYLHSMGKMHRDIKGA
NILLTEYGDVKLADFGVSAQITATINKRKSFIGTPYWMAPEVAAVERKGG
YNQLCDIWACGITAIELAELQPPMFDLHPMRALFLMSKSGFKPPTLNNKD
KWSPTFHNFIKTALTKNPKKRPTAERLLQHPFVQCEMSLRVAKELLQKYQ
SPNPQFYYYLDGDEESVAGVPQRIASKMTSRTNGVPAQNHTLKTGMTTNS
TWNERSSSPETLPSDMSLLQYIDEELKLRATLPLNNDTKDPLGAECSCSS
HNGGAAGGGGGGGVGVGAGGGAANGSSSSSGGATVGTTHHQHQQHHQHHH
HPNHLHQHQSHQLPQQQQQQSHQAAQQEHHHHHPMTSSISSGLVTANANN
ISSSASLASMPGLSAYLGMRSHVVGHMGMGFGNGLGMSNLRTTSIDADDD
ELVAADVAMNNAAAAVPGNGSGAGNGSGSCCSASAAHYLRYSSNYRSAAA
AQASQAAHPHVNSNSNSNNGHGHAPITSSISSSASSASFYNRLLLLDNSS
GDAVVGGNGGGSSNISSSGGASSGTNGLLDKHELDALPQAATKSAGDGFS
HSNSPTANSGAGATGSRDNDGPSSSNSSHLYQNLLRSSSGETPAGSSSAG
NNCDYRHENNQNGLEDSPRRHSSMDQLIGLLENMGKSPRTRSLSDGGTQD
DDEAEKEAQPDLLNNTPPVPPKRSHKRRHTPPRPISNGLPPTPKVHMGAC
FSKIFNCCPLRVHCTASWIHPETRDQHLLIGAEEGIYNLNMNELHDAAID
QLFPRRTTWLYVIKDVLMSLSGKSCQLYRHDLVALHSKQTVRFSLHMNKI
PERLVPRKFALTTKVPDTKCCTQCCVTRNPYNGYKYLCGATPSGIFLMQW
YDPLNKFMLLKQCEWPAISIQGGGHGCVQNGHTPVFEMIITPELEYPIVC
TGVRKAMNGCLKLELINMNSASWFHSEDLEYDAMATMVPRRDLLKVVRVH
QVEKDAILVCYGNLIQVVTLQGNPKQHKKMVSQLNFDFNVDSIVCLPDSV
LAFHKHGMQGKSLRNGEVTQEIKDMSRTYRLLGSDKVVALESQLLRTGSL
GSEEGHDLYILAGHEASY
SEQ ID NO: 2
CATTTGTGGGCTGCGCGTTGACGAAGTGCATGTGAAATTTTTCGTGTTCG
TAGGACAATTGTTAATTATCCACTGGCCGAATTTGCCCCGCAATCACGTG
TCGGTGTAATATGTGAACGCGTCCAAAATGCATCTCGATCCACAAAAGTA
TCTAAATTTCGCCTGCTACGTGTGCTAATCAGTCAAACCCAAAAAACGAA
TAAAGCAGCGTCAAGAGTAAGAGTGTCAATCAAATATTAAGAGTATACCC
ACCCAGTAAAGAGAGGTCCAGCCAAAAGGACTCCATCGCCTCGAACCGAA
AGCGGAAGCGGAAGCGGAAACGGCAACGGCATACGGATACGGAAGCTGCC
AAGATGGCCGCCGCCCACAGTCACCACAATGCCAATATGCTCAGCTCGGA
CATCTCTCGCCGAAATCCGCAGGACGAGTACGAACTCATCCAAAAGATTG
GCTCGGGAACCTACGGCGACGTCTATAAGGCGAAACGAATACAGAGCAAT
GAGCTGGCTGCCATCAAGGTCATCAAGCTGGAGCCTTCCGACGATATACA
GATTATCCAGCAGGAGATTATCATGATGAGGGACTGCCGCCATCCGAACA
TCATCGCCTACTACGGTTCGTACCTGCGCAGGGATAAACTCTGGATCTGC
ATGGAGTTTTGTGGTGGTGGCAGTCTGCAGCACATTTACCAAGTGACCGG
TCCCCTCACCGAAGTCCAGATCGCGTACATGTGCCGAGAGACTCTCAAGG
GACTCGAGTACCTGCACTCCATGGGAAAAATGCACCGGGACATCAAGGGC
GCAAATATCCTGTTGACGGAGTACGGCGATGTCAAGCTGGCGGATTTCGG
CGTTTCAGCTCAGATCACGGCCACAATTAACAAACGCAAGAGCTTCATAG
GTACGCCCTACTGGATGGCTCCTGAGGTGGCAGCCGTGGAACGCAAGGGT
GGCTATAATCAACTATGTGATATTTGGGCCTGCGGCATAACCGCAATCGA
ACTGGCTGAACTGCAACCGCCGATGTTCGATTTGCACCCGATGCGCGCCC
TATTCCTGATGTCGAAGAGCGGCTTTAAGCCGCCCACTCTGAACAACAAG
GACAAGTGGAGCCCCACGTTCCACAACTTCATCAAGACGGCGCTGACGAA
GAATCCGAAGAAGCGACCCACCGCCGAGCGCCTGCTGCAGCATCCCTTCG
TCCAGTGCGAGATGTCCTTGCGGGTGGCCAAGGAGCTGCTGCAGAAGTAC
CAGAGTCCCAACCCGCAGTTCTACTACTATCTCGATGGCGATGAGGAGTC
TGTGGCAGGAGTGCCACAACGCATTGCCAGCAAAATGACGTCACGCACCA
ATGGCGTGCCAGCGCAAAATCACACACTAAAAACAGGCATGACGACGAAC
TCCACGTGGAATGAGCGATCTTCTAGTCCCGAAACGTTACCCAGTGACAT
GAGCCTCTTACAATATATTGATGAGGAGCTGAAGCTAAGAGCGACCTTGC
CACTGAACAACGACACCAAAGATCCACTCGGCGCCGAGTGCAGCTGCTCC
TCCCACAATGGAGGAGCCGCCGGAGGAGGAGGAGGAGGAGGAGTTGGAGT
AGGAGCAGGCGGAGCAGCCGCGAACGGCAGCAGCAGCAGCAGCGGAGGCG
CAACAGTCGGCACCACTCATCATCAGCACCAACAGCACCACCAGCATCAC
CACCATCCGAATCATCTGCATCAGCATCAGTCCCATCAATTGCCGCAACA
GCAGCAGCAGCAGTCACATCAGGCGGCACAGCAGGAACACCATCACCACC
ATCCAATGACCTCGTCCATATCCAGCGGCTTGGTGACCGCCAATGCCAAC
AACATCAGCTCGTCCGCCTCGCTGGCCTCCATGCCAGGTCTCAGTGCCTA
CCTGGGCATGCGCTCCCATGTGGTTGGCCACATGGGTATGGGATTTGGCA
TGGGTCTCGGCATGTCCAACCTGCGCACCACCAGCATCGATGCCGACGAC
GATGAACTGGTGGCTGCCGATGTGGCCATGAATAATGCTGCTGCCGCCGT
TCCTGGGAACGGATCCGGTGCTGGCAACGGTTCCGGATCCGGATGCTCCG
CCTCTGCGGCGCACTATCTGCGTTACAGCAGCAACTACCGATCCGCCGCC
GCGGCTCAGGCATCGCAGGCGGCACATCCGCACGTTAACTCCAATTCGAA
TTCGAACAACGGCCATGGCCATGCGCCGATCACATCCAGCATCTCGTCCT
CTGCGTCATCAGCTTCGTTTTATAATCGCCTCCTACTCCTCGATAATTCC
AGCGGCGATGCAGTCGTAGGCGGTAACGGAGCCGGCAGCAGCAACATAAG
CAGCAGCGGAGGAGCATCAAGTGGCACCAATGGCCTCCTGGATAAGCACG
AGCTAGATGCTCTTCCCCAAGCGGCAACAAAGTCGGCCGGCGATGGCTTC
TCGCATTCCAATAGCCCAACGGCGAATTCCGGTGCGGGAGCGACCGGATC
CAGGGATAACGATGGACCCTCGTCGAGCAACTCTTCGCATCTCTACCAGA
ACCTGCTGAGAAGCAGCTCTGGTGAAACGCCAGCGGCATCGAGCTCGGCG
GGCAATAACTGTGATTACCGGCATGAGAACAACCAGAATGGTCTGGAGGA
CTCGCCTCGACGCCATAGCTCCATGGATCAGCTGATTGGGTTGCTCGAGA
ACATGGGCAAGTCTCCGAGGACACGCAGCCTGAGCGATGGTGGCACCCAG
GACGATGACGAACCGGAGAAGGAGGCGCAACCGGATCTGCTGAACAACAC
CCCACCGGTACCACCGAAGCGCTCCCACAAACGCCGTCACACGCCACCTC
GACCCATCTCCAACGGACTGCCGCCCACACCCAAGGTGCACATGGGCGCC
TGCTTCTCCAAGATCTTCAACGGTTGCCCACTGCGCGTCCACTGCACCGC
CTCGTGGATTCATCCGGAGACGCGGGACCAGCATCTGCTGATCGGCGCCG
AGGAGGGCATCTACAACCTCAACATGAACGAACTGCATGACGCGGCCATC
GATCAGTTGTTCCCGCGCCGCACCACCTGGTTGTATGTCATCAAGGATGT
GCTCATGAGTTTGTCAGGAAAATCCTGCCAGCTCTACAGGCACGACCTGG
TGGCCCTGCACTCCAAGCAGACGGTACGCTTCTCGCTGCACATGAACAAG
ATCCCGGAGCGACTGGTGCCGCGCAAGTTCGCTCTCACCACCAAGGTGCC
GGACACAAAGTGCTGCACACAGTGCTGTGTCACACGGAATCCGTATAACG
GATACAAATATCTCTGTGGGGCGACACCCAGCGGCATTTTCCTGATGCAG
TGGTACGATCCACTGAACAAGTTTATGCTGCTCAAGCAGTGCGAGTGGCC
AGCCATCAGCATTCAGGGTGGTGGTCATGGCTGCGTTCAGAACGGACATA
CGCCCGTATTCGAGATGATTATCACGCCGGAGTTGGAGTACCCGATTGTG
TGTACGGGTGTGCGAAAGGCCATGAACGGCTGCCTCAAGCTGGAGCTGAT
CAACATGAACAGTGCCAGCTGGTTCCATTCGGAGGACCTGGAATATGACG
CCATGGCCACGATGGTGCCGCGACGCGACCTGCTGAAGGTGGTGCGTGTG
CACCAGGTGGAGAAGGACGCCATTCTGGTCTGCTACGGCAATCTGATTCA
GGTGGTCACCCTGCAGGGCAATCCCAAGCAGCACAAGAAGATGGTATCGC
AGCTCAACTTTGATTTCAACGTGGACAGCATTGTTTGTCTACCGGACAGC
GTTTTGGCATTCCATAAGCACGGCATGCAGGGAAAGTCGTTGCGCAATGG
CGAGGTGACGCAGGAGATCAAGGACATGAGTCGCACCTACAGGCTGCTGG
GCAGCGATAAGGTTGTGGCCTTGGAGAGCCAGCTGCTGAGGACCGGCTCC
CTGGGCAGCGAGGAGGGACACGATCTGTACATTCTGGCCGGGCACGAGGC
TAGCTACTAAGCTAATGGGCGCCCGCCCAATCCACTGTTATATTTTAAAT
ATAATCCGGTTCACCACACACATTTTAAAGGGCTGTCGCCGCCGTCGTCG
TCAACTATTAACTGTAATTTAATTACTCGTAGGCTAAGCAGTGAGTTCAC
TGATCATAGATGACTAGAGAGAGCCAGTTGCATGCCGAGCGTTCGATAGC
TTAGATTAGCGCAATTTAACAGCACCTACAAGCGCCGGTCTTCCAAGAAG
GGTTCCACACTACCATGCACACTGACGGGTATACTCGACCGCAATCGCTA
TCTTTAGATCTGCCGATCACTCGATATATATTGAACGTCTGCGTCTACCC
GATTCCGTTGTAGCTATATTTTTGTATTACAAAGAAATGCTTTAATTTAT
ACAACTAACCTTAATGAACTACAGTAATCGCTACGTCTAGTTATGTTAGA
GATTTGTGTAAACGCTTCACAAAGCCAATAGATACGTTTTCAAATAGTCA
GCCAGCAGGAGAATCCAAGTATACATACACTTAAACACGCAATTATCCAC
ACGATTCGAGACCGACTAAGGCAATTAGAACTAAACAATTATATTTAGTC
TATAGCAATTGTAAATCGCAATGTTTTTAGCTGGTTAAACTGCTTTCGAT
TGGCTCAAAACCGCAACGGAAAATCAAGAACGAGAAATTGAAGAATGCGA
TATAGGATGATACGGTTTGTTGGGAAACAAAACTAGTTAGTAAGACTTTT
CTAACCTAGTAACACGAATTCAAAACAAAATAAATGCAAATTAATGGTCT
GTATCTGTATGTTAAAAAAGTAAACTGATTGTCATAGTTACGGTTCTAGG
CTATAAGATAAGCGATGAAAACAGACAACTTTCCGAACAGCACATTTGTC
GATTGAGTTGTATAAATATTCTACTAGATGCTAAGTTATTTAGTTCATAC
GACAACAACAAAGCAAAAACCGAAAATTACAAACTAATACGGAGAAAAAC
AAACAGATAAACTCACAAAATTGTTGAACTCTAGCTTAGTGCAAAAATAA
AAAAAAAATACGTGAAAGAAA
SEQ ID NO: 3
MAAAHSHHNANMLSSDISRRNPQDEYELIQKIGSGTYGDVYKAKRIQSNE
LAAIKVIKLEPSDDIQIIQQEIIMMRDCRHPNIIAYYGSYLRRDKLWICM
EFCGGGSLQDIYQVTGPLTEVQIAYMCRETLKGLEYLHSMGKMHRDIKGA
NILLTEYGDVKLADFGVSAQITATINKRKSFIGTPYNMAPEVAAVERKGG
YNQLCDIWACGITAIELAELQPPMFDLNPMRALFLMSKSGFKPPTLNNKD
KWSPTFHNFIKTALTKNPKKRPTAERLLQHPFVQCEMSLRVAKELLQKYQ
SPNPQFYYYLDGDEESVAGVPQRIASKMTSRTNGVPAQNHTLKTGMTTNS
TWNERSSSPETLPSDMSLLQYIDEELKLSGDAVVGGNGGGSSNISSSGGA
SSGTNGLLDKHELDALPQAATKSACDGFSHSNSPTANSGAGATGSRDNDG
PSSSNSSHLYQNLLRSSSGETPAGSSSAGNNCDYRHENNQNGLEDSPRRH
SSMDQLIGLLENMGKSPRTPSLSDGGTQDDDEAEKEAQPDLLNNTPPVPP
KRSHKRRHTPPRPISNGLPPTPKVHMGACFSKIFNGCPLRVHCTASWIHP
ETRDQHLLIGAEEGIYNLNMNELHDAAIDQLFPRRTTWLYVIKDVLMSLS
GKSCQLYRHDLVALHSKQTVRFSLHMNKIPERLVPRKFALTTKVPDTKCC
TQCCVTRNPYNGYKYLCGATPSGIFLMQWYDPLNKFMLLKQCEWPAISIQ
GGGHGCVQNGHTPVFEMIITPELEYPIVCTGVRKAMNGCLKLELINMNSA
SWFHSEDLEYDAMATMVPRRDLLKVVRVHQVEKDAILVCYGNLIQVVTLQ
GNPKQHKKMVSQLNFDFNVDSIVCLPDSVLAFHKHGMQGKSLRNGEVTQE
IKDMSRTYRLLGSDKVVALESQLLRTGSLGSEEGHDLYILAGHEASY
SEQ ID NO: 4
CATTTGTGGGCTGCGCGTTGACGAAGTGCATGTGAAATTTTTCGTGTTCG
TAGGACAATTGTTAATTATCCACTGGCCGAATTTGCCCCGCAATCACGTG
TCGGTGTAATATGTGAACGCGTCCAAAATGCATCTCGATCCACAAAAGTA
TCTAAATTTCGCCTGCTACGTGTGCTAATCAGTCAAACCCAAAAAACGAA
TAAAGCAGCGTCAAGAGTAAGAGTGTCAATCAAATATTAAGAGTATACCC
ACCCAGTAAAGAGAGGTCCAGCCAAAAGGACTCGATCGCCTCGAACCGAA
AGCGGAAGCGGAAGCGGAAACGGCAACGGCATACGGATACGGAAGCTGCC
AAGATGGCCGCCGCCCACAGTCACCACAATGCCAATATGCTCAGCTCGGA
CATCTCTCGCCGAAATCCGCAGGACGAGTACGAACTCATCCAAAAGATTG
GCTCGGGAACCTACGGCGACGTCTATAAGGCGAAACGAATACAGAGCAAT
GAGCTGGCTGCCATCAAGGTCATCAAGCTGGAGCCTTCCGACGATATACA
GATTATCCAGCAGGAGATTATCATGATGAGGGACTGCCGCCATCCGAACA
TCATCGCCTACTACGGTTCGTACCTGCGCAGGGATAAACTCTGGATCTGC
ATGGAGTTTTGTGGTGGTGGCAGTCTGCAGGACATTTACCAAGTGACCGG
TCCCCTCACCGAAGTCCAGATCGCGTACATGTGCCGAGAGACTCTCAAGG
GACTGGAGTACCTGCACTCCATGGGAAAAATGCACCGGGACATCAAGGGC
GCAAATATCCTGTTGACGGAGTACGGCGATGTCAAGCTGGCGGATTTCGG
CGTTTCAGCTCAGATCACGGCCACAATTAACAAACGCAAGAGCTTCATAG
GTACGCCCTACTGGATGGCTCCTGAGGTGGCAGCCGTGGAACGCAAGGGT
GGCTATAATCAACTATGTGATATTTGGGCCTGCGGCATAACCGCAATCGA
ACTGGCTGAACTGCAACCGCCGATGTTCGATTTGCACCCGATGCGCGCCC
TATTCCTGATGTCGAAGAGGGGCTTTAAGCCGCCCACTCTGAACAACAAG
GACAAGTGGAGCCCCACGTTCCACAACTTCATCAAGACGGCGCTGACGAA
GAATCCGAAGAAGCGACCCACCGCCGAGCGCCTGCTGCAGCATCCCTTCG
TCCAGTGCGAGATGTCCTTGCGGGTGGCCAAGGAGCTGCTGCAGAAGTAC
CAGAGTCCCAACCCGCAGTTCTACTACTATCTCGATGGCGATGAGGAGTC
TGTGGCAGGAGTGCCACAACGCATTGCCAGCAAAATGACGTCACGCACCA
ATGGCGTGCCAGCGCAAAATCACACACTAAAAACAGGCATGACGACGAAC
TCCACGTGGAATGAGCGATCTTCTAGTCCCGAAACGTTACCCAGTGACAT
GAGCCTCTTACAATATATTGATGAGGAGCTGAAGCTAAGCGGCGATGCAG
TCGTAGGCGGTAACGGAGGCGGCAGCAGCAACATAAGCAGCAGCGGAGGA
GCATCAAGTGGCACCAATGGCCTCCTGGATAAGCACGAGCTAGATGCTCT
TCCCCAAGCGGCAACAAAGTCGGCCGCCGATGGCTTCTCGCATTCCAATA
GCCCAACGGCGAATTCCGGTGCGGGACCGACCGGATCCAGGGATAACGAT
GGACCCTCGTCGAGCAACTCTTCGCATCTCTACCAGAACCTGCTGAGAAG
CAGCTCTGGTGAAACGCCAGCGGGATCGAGCTCGGCGGGCAATAACTGTG
ATTACCGGCATGAGAACAACCAGAATGGTCTGGAGGACTCGCCTCGACGC
CATAGCTCCATGGATCAGCTGATTGGGTTGCTCGAGAACATGGGCAAGTC
TCCGAGGACACGCAGCCTGAGCGATGGTGGCACCCAGGACGATGACGAAG
CGGAGAAGGAGGCGCAACCGGATCTGCTGAACAACACCCCACCGGTACCA
CCGAAGCGCTCCCACAAACGCCGTCACACGCCACCTCGACCCATCTCCAA
CGGACTGCCGCCCACACCCAAGGTGCACATGGGCCCCTGCTTCTCCAAGA
TCTTCAACGGTTGCCCACTGCGCGTCCACTGCACCGCCTCGTGGATTCAT
CCGGAGACGCGGGACCAGCATCTGCTGATCGGCGCCGAGGAGGGCATCTA
CAACCTCAACATGAACGAACTGCATGACGCGGCCATCGATCAGTTGTTCC
CGCGCCGCACCACCTGGTTGTATGTCATCAAGGATGTGCTCATGAGTTTG
TCAGGAAAATCCTGCCAGCTCTACAGGCACGACCTGGTGGCCCTGCACTC
CAAGCAGACGGTACGCTTCTCGCTGCACATGAACAAGATCCCGGAGCGAC
TGGTGCCGCGCAAGTTCGCTCTCACCACCAAGGTGCCGGACACAAAGTGC
TGCACACAGTGCTGTGTCACACGGAATCCGTATAACGGATACAAATATCT
CTGTGGGGCGACACCCAGCGGCATTTTCCTGATGCAGTGGTACGATCCAC
TGAACAAGTTTATGCTGCTCAAGCAGTGCGACTGGCCAGCCATCAGCATT
CAGGGTGGTGGTCATGGCTGCGTTCAGAACGGACATACGCCCGTATTCGA
GATGATTATCACGCCGCAGTTGGAGTACCCGATTGTGTGTACGGGTGTGC
GAAAGGCCATGAACGGCTGCCTCAAGCTGGAGCTGATCAACATGAACAGT
GCCAGCTGGTTCCATTCGGAGGACCTGGAATATGACGCCATGGCCACGAT
GGTGCCGCGACGCGACCTGCTGAAGGTGGTGCGTGTGCACCAGGTGGAGA
AGGACGCCATTCTGGTCTGCTACGGCAATCTGATTCAGGTGGTCACCCTG
CAGGGCAATCCCAAGCAGCACAAGAAGATGGTATCGCAGCTCAACTTTGA
TTTCAACGTGGACAGCATTGTTTGTCTACCGGACAGCGTTTTGGCATTCC
ATAAGCACGGCATGCAGGGAAAGTCGTTGCGCAATGGCGAGGTGACGCAG
GAGATCAAGGACATGAGTCGCACCTACAGGCTGCTGGGCAGCGATAAGGT
TGTGGCCTTGGAGAGCCAGCTGCTGAGGACCGGCTCCCTGGGCAGCGAGG
AGGGACACGATCTGTACATTCTGGCCGGGCACGAGGCTAGCTACTAAGCT
AATGGGCGCCCGCCCAATCCACTGTTATATTTTAAATATAATCCGGTTCA
CCACACACATTTTAAAGGGCTGTCGCCGCCGTCGTCGTCAACTATTAACT
GTAATTTAATTACTCCTAGGCTAAGCAGTGAGTTCACTGATCATAGATGA
CTAGAGAGAGCCAGTTGCATGCCGAGCGTTCGATAGCTTAGATTAGCGCA
ATTTAACAGCACCTAGAAGCGGCGGTCTTCGAAGAAGGGTTCCACACTAC
CATGCACACTGACGGGTATACTCGACCCGAATCGCTATCTTTAGATCTGC
CGATCACTCGATATATATTGAACGTCTGCGTCTACCCGATTCCGTTGTAG
CTATATTTTTGTATTACAAAGAAATGCTTTAATTTATACAACTAACCTTA
ATGAACTACAGTAATCGCTACGTCTAGTTATGTTAGAGATTTGTGTAAAC
GCTTCACAAAGCCAATAGATACGTTTTCAAATAGTCAGCCAGCAGGAGAA
TCCAAGTATACATACACTTAAACACGCAATTATCCACACGATTCGAGACC
GACTAAGGCAATTAGAACTAAACAATTATATTTAGTCTATAGCAATTGTA
AATCGCAATGTTTTTAGCTGGTTAAACTGCTTTGGATTGGCTCAAAACCG
CAACGGAAAATCAAGAACGAGAAATTGAAGAATGCGATATAGGATGATAC
GGTTTGTTGGGAAACAAAACTAGTTAGTAAGACTTTTCTAACCTAGTAAC
ACGAATTCAAAACAAAATAAATGCAAATTAATGGTCTGTATCTGTATGTT
AAAAAAGTAAACTGATTGTCATAGTTACGGTTCTAGGCTATAAGATAAGC
GATGAAAACACAGAACTTTCCGAACAGCACATTTGTCGATTGAGTTGTAT
AAATATTCTACTAGATGCTAAGTTATTTAGTTCATACGACAACAACAAAG
CAAAAACCGAAAATTACAAACTAATACGGAGAAAAACAAACAGATAAACT
CACAAAATTGTTGAACTCTAGCTTAGTGCAAAAATAAAAAAAAAATACGT
GAAAGAAA

What is claimed is:

1. An isolated nucleic acid encoding a polypeptide comprising an amino acid sequence having at least 95% identity to SEQ ID NO:1 or 3.

2. The isolated nucleic acid of claim 1 encoding a polypeptide comprising the amino acid sequence of SEQ ID NO:1 or 3.

3. The isolated nucleic acid of claim 1 comprising a nucleic acid sequence having at least 95% identity to about 500 contiguous nucleotides selected from SEQ ID NO:2 or 4 or the complement thereof.

4. The isolated nucleic acid of claim 1 comprising the nucleic acid sequence of SEQ ID NO:2 or 4 or the complement thereof.

5. An isolated polypeptide comprising an amino acid sequence having at least 95% identity to SEQ ID NO:1 OR 3.

6. The isolated polypeptide of claim 5, wherein the polypeptide comprises the amino acid sequence of SEQ ID NO:1 OR 3.

7. A vector comprising the isolated nucleic acid of claim 1, wherein the encoded polypeptide is capable of phosphorylating myosin light chain.

8. The vector of claim 7 comprising the nucleic acid sequence of SEQ ID NO:2 or 4.

9. The vector of claim 7, wherein the nucleic acid is operably linked to a transcriptional regulatory sequence.

10. The vector of claim 7, wherein said vector is selected from the group comprising a plasmid, a cosmid, a virus, and a bacteriophage.

11. The vector of claim 7, wherein a polypeptide comprising SEQ ID NO:1 OR 3 is expressed by a cell transformed with said vector.

12. An isolated host cell comprising the nucleic acid of claim 1.

13. An isolated host cell comprising the vector of claim 7.

14. An antibody that specifically binds to a polypeptide comprising an amino acid sequence of SEQ ID NO:1 OR 3.

15. The antibody of claim 14, wherein the antibody is polyclonal.

16. The antibody of claim 14, wherein the antibody is monoclonal.

17. The antibody of claim 14, wherein the antibody is single chain monoclonal.

18. The antibody of claim 14, wherein the antibody is recombinant.

19. The antibody of claim 14, wherein the antibody is chimeric.

20. The antibody of claim 14, wherein the antibody is humanized.

21. The antibody of claim 14, wherein the antibody is mammalian.

22. The antibody of claim 14, wherein the antibody is human.

23. A method of screening for an agent capable of modulating happyhour activity, comprising: a) contacting said agent with a cell that expresses a happyhour polypeptide; and b) assessing a biological activity of the happyhour in the cell.

24. The method of claim 23, wherein the biological activity is selected from modulation of ethanol sedation and modulation of EGFR/ERK signaling.

25. The method of claim 23, wherein said cell is selected from the group of cells consisting of Insulin producing cells (IPC), Dopaminergic neurons (DA neurons), Serotonergic neurons (5HT neurons), Antennal lobe (AL) cells, Antenno-mechanosensory center (AMC) cells, Subesophageal ganglion (SEG) cells, Central complex (CC) cells; Lateral protocerebrum (LPC) cells, Mushroom Body (MB) cells, Dorsal giant interneurons (DGI), Ellipsoid body (EB) cells, Ventral lateral neurons (LNv neurons), and Optic lobes (OL) cells.

26. The method of claim 23, wherein said cell is selected from a Drosophila cell, a mouse cell, a rat cell, or a human cell.

27. A method of screening for an agent capable of modulating drug-induced impairment, comprising: a) contacting a cell with said agent capable of modulating EGFR; b) assessing a biological activity of EGFR; and c) correlating the biological activity of EGFR with a biological activity of happyhour in the cell.

28. The method of claim 27, wherein said cell is selected from the group of cells consisting of Insulin producing cells (IPC), Dopaminergic neurons (DA neurons), Serotonergic neurons (5HT neurons), Antennal lobe (AL) cells, Antenno-mechanosensory center (AMC) cells, Subesophageal ganglion (SEG) cells, Central complex (CC) cells; Lateral protocerebrum (LPC) cells, Mushroom Body (MB) cells, Dorsal giant interneurons (DGI), Ellipsoid body (EB) cells, Ventral lateral neurons (LNv neurons), and Optic lobes (OL) cells.

29. The method of claims 27, wherein said cell is selected from a Drosophila cell, a mouse cell, a rat cell, or a human cell.
